SEQUENCE LISTING (19 sequences)

SEQ ID NO: 1 - protein, 27 aa, synthetic construct
GALFLGFLGAAGSTMGAWSQPKSKRKV

SEQ ID NO: 2 - protein, 21 aa, synthetic construct
KETWWETWWTEWSQPKKKRKV

SEQ ID NO: 3 - protein, 20 aa, synthetic construct
GLWRALWRLLRSLWRLLWRA

SEQ ID NO: 4 - protein, 30 aa, synthetic construct
WEAKLAKALAKALAKHLAKALAKALKACEA

SEQ ID NO: 5 - protein, 20 aa, synthetic construct
GLFGAIAGFIENGWEGMIDG

SEQ ID NO: 6 - protein, 8 aa, synthetic construct
RRRRRRRR

SEQ ID NO: 7 - protein, 7 aa, synthetic construct
KLWVLPK

SEQ ID NO: 8 - protein, 8 aa, synthetic construct
KLWVLPKK

SEQ ID NO: 9 - protein, 18 aa, synthetic construct
RRANAALKAGELTKCILY

SEQ ID NO: 10 - protein, 7 aa, synthetic construct
TKKTLRT

SEQ ID NO: 11 - protein, 10 aa, synthetic construct
WREPSFMALS

SEQ ID NO: 12 - protein, 7 aa, synthetic construct
KIWKLPQ

SEQ ID NO: 13 - protein, 7 aa, synthetic construct
KIFVWPY

SEQ ID NO: 14 - protein, 7 aa, synthetic construct
KVWSLPQ

SEQ ID NO: 15 - protein, 20 aa, synthetic construct
RRANAALKAGELYKSILYGC

SEQ ID NO: 16 - protein, 18 aa, synthetic construct
RRANAALKAGELYKCILY

SEQ ID NO: 17 - protein, 20 aa, synthetic construct
  VARIANT 5..20: note = Residues may be deleted
RRRRRRRRRRRRRRRRRRRR

SEQ ID NO: 18 - protein, 8 aa, synthetic construct
  SITE 1: note = stearic acid-Arginine
RRRRRRRR

SEQ ID NO: 19 - protein, 8 aa, synthetic construct
  SITE 8: note = Lysine(azide)
KLWVLPKK

(19) United States
(12) Patent Application Publication
(10) Pub. No.: US 2026/0224507 A1
(43) Pub. Date: Aug. 6, 2026

(54) MULTI-FUNCTIONAL LIPID NANOPARTICLES AND USES THEREOF

(71) Applicant: UNIVERSITY OF TENNESSEE RESEARCH FOUNDATION, Memphis, TN (US)

(72) Inventors: Deidra JH MOUNTAIN, Memphis, TN (US); Phillip Connor WEST, Memphis, TN (US); Richard FISHER, Memphis, TN (US)

(21) Appl. No.: 19/153,189

(22) PCT Filed: Feb. 6, 2024
(86) PCT No.: PCT/US2024/014622

§ 371 Date: Aug. 1, 2025

Related U.S. Application Data
(60) Provisional application No. 63/483,375, filed on Feb. 6, 2023.

(51) Int. Cl. A61K 9/51; A61K 31/7105; A61K 31/711; A61K 47/24; A61K 47/28; A61P 9/00

(52) U.S. Cl. CPC A61K 9/5192; A61K 9/5123; A61K 9/5146; A61K 9/5169; A61K 31/7105; A61K 31/711; A61K 47/24; A61K 47/28; A61P 9/00

(57) ABSTRACT

The present disclosure relates to lipid nanoparticles including a lipid layer, a cell-penetrating peptide conjugated to the lipid layer, a collagen-targeting peptide conjugated to the lipid layer and a nucleic acid associated with a vascular disease or condition. The present disclosure also relates to methods of making and using the described lipid nanoparticles for treating vascular disease.

### CROSS REFERENCE TO RELATED APPLICATIONS

This PCT application claims priority to U.S. provisional application No. 63/483,375, filed on Feb. 6, 2023, which is hereby incorporated by reference in its entirety.

### STATEMENT AS TO FEDERALLY SPONSORED RESEARCH

This invention was made with government support under Contract number 2309031 awarded by National Science Foundation. The government has certain rights in the invention.

### BACKGROUND

Peripheral vascular disease (PVD) is a blood circulation disorder that causes blood vessels to narrow, block or spasm. Endovascular interventions are commonly used to treat PVD in a minimally invasive fashion, but mechanical injury to the diseased vessels is unavoidable. This damage often results in endothelial cell disruption, exposure of the subendothelial matrix, and underlying vascular smooth muscle cells and initiates vascular wall remodeling that oftentimes contributes to the development of secondary vascular pathologies, such as intimal hyperplasia (IH)-induced restenosis.

RNA therapeutics have emerged as a promising new class of medicine, holding great potential to treat a broad array of diseases including vascular diseases. However, RNA can be vulnerable to enzymatic degradation. Thus, the ultimate hurdle to clinical breakthrough of RNA therapeutics continues to be the development of a safe and effective delivery system that efficiently packages, protects, and delivers RNA to diseased cells and tissues.

### SUMMARY

Described herein are compositions of lipid nanoparticles and methods of making and using lipid nanoparticles.

In one aspect, the present disclosure provides a lipid nanoparticle comprising a lipid layer comprising a helper lipid, a PEG lipid, a structural lipid, or a combination thereof, a cell-penetrating peptide conjugated to the lipid layer, a collagen-targeting peptide conjugated to the lipid layer, and a nucleic acid associated with a vascular disease or condition.

In some embodiments, the nucleic acid is encapsulated in the lipid nanoparticle. In some embodiments, the nucleic acid comprises a DNA. In some embodiments, the nucleic acid comprises an RNA. In some embodiments, the nucleic acid comprises an siRNA.

In one aspect, the present disclosure provides a lipid nanoparticle comprising a lipid layer comprising a helper lipid, a PEG lipid, a structural lipid, or a combination thereof, a cell-penetrating peptide conjugated to the lipid layer, a collagen-targeting peptide conjugated to the lipid layer, and a biologically active ingredient that comprises one or more of antibiotics, stimulants, statins, b-receptor blockers, anti-hypertensives, anticoagulants, bronchodilators, corticosteroids, insulin, vaccines, immunosuppressants, interferons, antibodies, proteins, and peptides.

In some embodiments, the collagen-targeting peptide is conjugated with a lipid to form a collagen-targeting lipid. In some embodiments, the collagen-targeting lipid comprises about 0.5 mol % to about 15 mol % of the total lipid in the lipid nanoparticle. In some embodiments, the collagen-targeting lipid comprises about 1 mol % to about 10 mol % of the total lipid in the lipid nanoparticle. In some embodiments, the collagen-targeting lipid comprises a PEG lipid. In some embodiments, the collagen-targeting lipid comprises pegylated 1, 2-distearoyl-sn-glycero-3-phosphoethanolamine (DSPE). In some embodiments, the collagen-targeting peptide comprises a sequence of KLWVLPK, KIWKLPQ, KIFVWPY, KVWSLPQ, RRANAALKAGELYKCILY, RRANAALKAGELYKSILYGC, TKKTLRT, or WREPSFMALS. In some embodiments, the collagen-targeting peptide comprises a sequence of KLWVLPK.

In some embodiments, the cell-penetrating peptide is conjugated with a lipid or a hydrophobic moiety to form a cell-penetrating lipid. In some embodiments, the cell-penetrating peptide comprises about 0.5 mol % to about 15 mol % of the total lipid in the lipid nanoparticle. In some embodiments, the cell-penetrating peptide comprises about 5 mol % to about 10 mol % of the total lipid in the lipid nanoparticle. In some embodiments, the cell-penetrating peptide is conjugated with a lipid to form the cell-penetrating lipid. In some embodiments, the cell-penetrating peptide is conjugated with a PEG lipid (e.g., pegylated DSPE) to form the cell-penetrating lipid. In some embodiments, the cell-penetrating peptide is conjugated with a hydrophobic moiety to form the cell-penetrating lipid. In some embodiments, the hydrophobic moiety is a fatty acid, fatty alcohol, or fatty ester. In some embodiments, the cell-penetrating peptide is covalently attached to a stearic acid to form the cell-penetrating lipid. In some embodiments, the cell-penetrating peptide comprises polyarginine, polylysine, polyhistidine, penetratin and derivatives thereof, MPG peptide, Pep-1 peptide, CADY peptide, KALA peptide, HA2 peptide, histones, poly-β-amino acid esters, or combinations thereof. In some embodiments, the cell-penetrating peptide comprises polyarginine, polylysine, or polyhistidine. In some embodiments, the cell-penetrating peptide comprises polyarginine. In some embodiments, the polyarginine is octaarginine (R8). In some embodiments, the cell-penetrating peptide is conjugated with stearic acid to form a cell-penetrating lipid, and wherein the cell-penetrating lipid is STR-R8.

In some embodiments, the lipid nanoparticle comprises at least one of a monovalent cation or a multivalent cation. In some embodiments, the monovalent cation or multivalent cation is a divalent cation. In some embodiments, the divalent cation is selected from the group consisting of calcium (Ca2+), magnesium (Mg2+), ferrous (Fe2+), and combinations thereof. In some embodiments, the divalent cation is calcium (Ca2+). In some embodiments, the at least one of the monovalent cation or the multivalent cation has a concentration of about 5 mM to about 50 mM. In some embodiments, the concentration is about 10 mM to about 40 mM. In some embodiments, the concentration is about 10 mM to about 30 mM. In some embodiments, the concentration is about 5 mM to about 15 mM.

In some embodiments, the lipid nanoparticle comprises a helper lipid. In some embodiments, the helper lipid is a phospholipid. In some embodiments, the helper lipid is 1,2-dioleoyl-sn-glycero-3-phosphocholine (DOPC), 1,2-distearoyl-sn-glycero-3-phosphoethanolamine (DSPE), 1,2-distearoyl-sn-glycero-3-phosphocholine (DSPC), 1,2-dipalmitoyl-sn-glycero-3-phosphocholine (DPPC), 1,2-dimyristoyl-sn-glycero-phosphocholine (DMPC), 2-oleoyl-1-palmitoyl-sn-glycero-3-phosphocholine (POPC), 1,2-dioleoyl-sn-glycero-3-phosphoethanolamine (DOPE), sphingomyelin, phosphatidyl inositol (PI), or phosphatidic acid (PA). In some embodiments, the helper lipid has a plurality of hydrocarbon chains. In some embodiments, the helper lipid has a hydrocarbon tail including at least four carbon atoms. In some embodiments, the helper lipid has a hydrocarbon tail including at least 6 carbon atoms. In some embodiments, the helper lipid has a hydrocarbon tail including at least 8 carbon atoms. In some embodiments, the helper lipid has a hydrocarbon tail including at least 10 carbon atoms. In some embodiments, the helper lipid has one or more unsaturated hydrocarbons. In some embodiments, the helper lipid is DOPC. In some embodiments, the helper lipid comprises about 10 mol % to about 80 mol % of the total lipid in the nanoparticle.

In some embodiments, the lipid nanoparticle comprises a PEG lipid. In some embodiments, the PEG lipid comprises PEG-dilauroylglycerol, PEG-dimyristoylglycerol (PEG-DMG), PEG-dipalmitoylglycerol, PEG-distearoylglycero-phosphoethanolamine (PEG-DSPE), PEG-dipalmitoylglycerol, PEG-disterylglycerol, PEG-dilaurylglycamide, PEG-dimyristylglycamide, PEG-dipalmitoylglycamide, PEG-disterylglycamide, PEG-cholesterol, and PEG-DMB (3,4-Ditetradecoxylbenzyl-[omega]-methyl-poly(ethylene glycol) ether), 1,2-dimyristoyl-sn-glycero-3-phosphoethanolamine-N-[methoxy(polyethylene glycol)-2000]), PEG-DOPC, or a mixture thereof. In some embodiments, the PEG lipid is 1,2-distearoyl-sn-glycero-3-phosphoethanolamine-N-[methoxy(polyethyleneglycol)-2000](DSPE-PEG(2000)). In some embodiments, the PEG lipid comprises about 1 mol % to about 20 mol % of the total lipid in the nanoparticle.

In some embodiments, the lipid nanoparticle comprises a structural lipid. In some embodiments, the structural lipid comprises steroid, sterol, alkyl resoreinol, cholesterol or derivative thereof, fecosterol, sitosterol, ergosterol, campesterol, stigmasterol, brassicasterol, tomatidine, tomatine, ursolic acid, alpha-tocopherol, or a combination thereof. In some embodiments, the structural lipid is cholesterol. In some embodiments, the structural lipid comprises about 5 mol % to about 30 mol % of the total lipid in the nanoparticle.

In one aspect, the present disclosure provides a method of treating vascular disease in a subject in need thereof, comprising administering to the subject a lipid nanoparticle comprising a lipid layer, a cell-penetrating peptide conjugated to the lipid layer, a collagen-targeting peptide conjugated to the lipid layer and a therapeutically effective ingredient encapsulated in the lipid nanoparticle. In some embodiments, the therapeutically effective ingredient comprises DNA or RNA.

In some embodiments, the subject's blood alanine aminotransferase (ALT) level does not fluctuate more than 50% at 25 hours after the administering. In some embodiments, the subject's blood alanine aminotransferase (ALT) level does not fluctuate more than 25%, more than 50%, more than 75%, more than 100%, more than 150%, more than 250%, more than 500%, or more than 1000% at 25 hours after the administering. In some embodiments, the subject's blood alanine aminotransferase (ALT) level does not fluctuate more than 25%, more than 50%, more than 75%, more than 100%, more than 150%, more than 250%, more than 500%, or more than 1000% at 4 hours after the administering. In some embodiments, the subject's blood alanine aminotransferase (ALT) level does not fluctuate more than 25%, more than 50%, more than 75%, more than 100%, more than 150%, more than 250%, more than 500%, or more than 1000% at 48 or 72 hours after the administering.

In some embodiments, the subject's blood aspartate aminotransferase (AST) level does not fluctuate more than 50% at 25 hours after the administering. In some embodiments, the subject's blood aspartate aminotransferase (AST) level does not fluctuate more than 25%, more than 50%, more than 75%, more than 100%, more than 150%, more than 250%, more than 500%, or more than 1000% at 25 hours after the administering. In some embodiments, the subject's blood aspartate aminotransferase (AST) level does not fluctuate more than 25%, more than 50%, more than 75%, more than 100%, more than 150%, more than 250%, more than 500%, or more than 1000% at 4 hours after the administering. In some embodiments, the subject's blood aspartate aminotransferase (AST) level does not fluctuate more than 25%, more than 50%, more than 75%, more than 100%, more than 150%, more than 250%, more than 500%, or more than 1000% at 48 or 72 hours after the administering.

In another aspect, the present disclosure provides a method of preparing lipid nanoparticles, comprising combining a hydrophobic mixture with an aqueous solution, thereby forming the lipid nanoparticle and isolating the lipid nanoparticles. The hydrophobic mixture comprises one or more lipids, where the one or more lipids comprise a helper lipid, a PEG lipid, a structural lipid, or a combination thereof, a cell-penetrating peptide and a collagen-targeting peptide. The aqueous solution comprises a cation and a biological active ingredient, wherein the biological active ingredient is associated with a vascular disease or condition, or some condition thereby related by atypical collagen exposure or expression.

In some embodiment, the biological active ingredient comprises nucleic acid. In some embodiment, the nucleic acid comprises an siRNA. In some embodiment, the cation comprises at least one of a monovalent cation or a multivalent cation.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

Various aspects of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 is an illustrative representation of a multi-functional lipid nanoparticle encapsulated with a biologically active ingredient, according to some embodiments of the present disclosure.

FIG. 2 is an illustrative representation of another multi-functional lipid nanoparticle encapsulated with a biologically active ingredient, according to some embodiments of the present disclosure.

FIG. 3 is an illustrative representation of another multi-functional lipid nanoparticle encapsulated with multi-functional lipid nanoparticles and a biologically active ingredient, according to some embodiments of the present disclosure.

FIG. 4 is an illustrative representation of multi-functional lipid nanoparticles delivered to damaged endothelium with exposed collagen fibers, according to some embodiments of the present disclosure.

FIG. 5 is the Mass spectrum and chemical structure of DSPE-PEG-DBCO lipid with a cyclooctyne modification, as described in Example 1.

FIG. 6 is the Mass spectrum and chemical structure of an example collagen-targeting peptide (CTP), as described in Example 1.

FIG. 7 is the Mass spectrum and chemical structure of an example collagen-targeting peptide-conjugated lipid, as described in Example 1.

FIG. 8 illustrates the fluorescent images of human aortic smooth muscle cells (HASMC) after incubating with base lipid nanoparticles PLPs, collagen-targeting peptide-conjugated lipid nanoparticles CTP-PLPs, cell-penetrating peptide-conjugated lipid nanoparticles R8-PLPs and multi-functional lipid nanoparticles conjugated with collagen-targeting peptide and cell-penetrating peptide CTP-R8-PLPs, according to some embodiments of the present disclosure.

FIG. 9 illustrates the quantification of cellular association of base lipid nanoparticles PLPs, collagen-targeting peptide-conjugated lipid nanoparticles CTP-PLPs, cell-penetrating peptide-conjugated lipid nanoparticles R8-PLPs and multi-functional lipid nanoparticles conjugated with collagen-targeting peptide and cell-penetrating peptide CTP-R8-PLPs, according to some embodiments of the present disclosure.

FIG. 10A illustrates fluorescent images of human vessel explants after perfused with base lipid nanoparticles PLPs, collagen-targeting peptide-conjugated lipid nanoparticles CTP-PLPs, cell-penetrating peptide-conjugated lipid nanoparticles R8-PLPs and multi-functional lipid nanoparticles conjugated with collagen-targeting peptide and cell-penetrating peptide CTP-R8-PLPs.

FIG. 10B illustrates the quantification of lipid nanoparticles binding to the human vessel explants.

FIG. 11 illustrates the lipid nanoparticle binding to non-injured vessels via localized vascular infusion using PBS, base lipid nanoparticles PLPs, cell-penetrating peptide-conjugated lipid nanoparticles R8-PLPs and multi-functional lipid nanoparticles conjugated with collagen-targeting peptide and cell-penetrating peptide CTP-R8-PLPs.

FIG. 12 illustrates the lipid nanoparticle binding to BA-injured vessels via localized vascular infusion using base lipid nanoparticles PLP, collagen-targeting peptide-conjugated lipid nanoparticles CTP-PLP, cell-penetrating peptide-conjugated lipid nanoparticles R8-PLP and multi-functional lipid nanoparticles conjugated with collagen-targeting peptide and cell-penetrating peptide CTP-R8-PLP.

FIG. 13A illustrates fluorescent images of non-injured and BA-injured vessels via localized vascular infusion using base lipid nanoparticles PLPs, cell-penetrating peptide-conjugated lipid nanoparticles R8-PLPs and multi-functional lipid nanoparticles conjugated with collagen-targeting peptide and cell-penetrating peptide CTP-R8-PLPs.

FIG. 13B illustrates the lipid nanoparticle binding to non-injured and BA-injured vessels via localized vascular infusion using PBS, base lipid nanoparticles PLPs, cell-penetrating peptide-conjugated lipid nanoparticles R8-PLPs and multi-functional lipid nanoparticles conjugated with collagen-targeting peptide and cell-penetrating peptide CTP-R8-PLPs.

FIG. 14 illustrates siRNA encapsulation efficiency of lipid nanoparticles PLPs, collagen-targeting peptide-conjugated lipid nanoparticles CTP-PLPs and multi-functional lipid nanoparticles conjugated with collagen-targeting peptide and cell-penetrating peptide CTP-R8-PLPs.

FIG. 15 illustrates the GAPDH gene silencing effect using control, collagen-targeting peptide-conjugated lipid nanoparticles CTP-PLPs and multi-functional lipid nanoparticles conjugated with collagen-targeting peptide and cell-penetrating peptide CTP-R8-PLPs encapsulated with siRNA.

FIG. 16A illustrates a rodent that underwent balloon angioplasty prior to lipid nanoparticle perfusion.

FIG. 16B illustrates localized lipid nanoparticle LNP perfusion post balloon angioplasty.

FIG. 16C illustrates the systemic blood flow is restored after lipid nanoparticle LNP perfusion.

FIG. 17A illustrates fluorescent images of BA-injured vessels via localized vascular infusion using Sham control, CTP-R8-PLP, and SM102.

FIG. 17B illustrates the lipid nanoparticle binding to BA-injured vessels via localized vascular infusion using multi-functional lipid nanoparticles conjugated with collagen-targeting peptide and cell-penetrating peptide CTP-R8-PLP and SM102.

FIG. 18A illustrates fluorescent images of BA-injured vessels via localized vascular infusion using Sham control, CTP-R8-PLP with a ratio of lipid to siRNA of 100:1, and a ratio of 20:1.

FIG. 18B illustrates the lipid nanoparticle binding to BA-injured vessels via localized vascular infusion using unloaded CTP-R8-PLP, CTP-R8-PLP with a ratio of lipid to siRNA of 100:1, and with a ratio of 20:1.

FIG. 19 shows body weight measurements of treatment groups across study days.

FIG. 20 shows body weight change of treatment groups across study days, where body weight of each group on Day 0 was used as initial value.

FIG. 21A depicts body weight for each member in the treatment group 1 across study days.

FIG. 21B depicts body weight for each member in the treatment group 2 across study days.

FIG. 21C depicts body weight for each member in the treatment group 3 across study days.

FIG. 21D depicts body weight for each member in the treatment group 4 across study days.

FIG. 21E depicts body weight for each member in the treatment group 5 across study days.

FIG. 21F depicts body weight for each member in the treatment group 6 across study days.

FIG. 22 illustrates alanine aminotransferase (ALT) measurement of the treatment groups.

FIG. 23 illustrates aspartate aminotransferase (AST) measurement of the treatment groups.

### DETAILED DESCRIPTION

Described herein are compositions of multi-functional lipid nanoparticles, and methods of making and using multi-functional lipid nanoparticles for treating vascular disease. The present disclosure relates to a lipid nanoparticle comprising a lipid layer comprising a helper lipid, a PEG lipid, a structural lipid, or a combination thereof, a cell-penetrating peptide conjugated to the lipid layer, a collagen-targeting peptide conjugated to the lipid layer, and a nucleic acid associated with a vascular disease or condition. The disclosure also relates to a method of treating vascular disease in a subject in need thereof, comprising administering to the subject a lipid nanoparticle comprising a lipid layer, a cell-penetrating peptide conjugated to the lipid layer, a collagen-targeting peptide conjugated to the lipid layer, and a therapeutically effective ingredient encapsulated in the lipid nanoparticle. The disclosure also relates to a method of preparing lipid nanoparticles comprising combining a hydrophobic mixture with an aqueous solution, where the hydrophobic mixture comprises one or more lipids, where the one or more lipids comprise a helper lipid, a PEG lipid, a structural lipid, or a combination thereof, a cell-penetrating peptide, and a collagen-targeting peptide, where the aqueous solution comprises a cation and a biological active ingredient, wherein the biological active ingredient is associated with a vascular disease or condition, thereby forming the lipid nanoparticles, and isolating the lipid nanoparticles.

Endovascular interventions are commonly used to ameliorate the effects of peripheral vascular disease in a minimally invasive fashion, but resulting mechanical injury to the diseased vessel is unavoidable. This damage often results in intimal denudation and exposure of the sub-endothelial matrix, and initiates vascular remodeling that contributes to development of secondary vascular pathologies and the need for repeated surgical interventions. The lipid nanoparticles as described herein provide various functions in diagnosis, drug delivery and monitoring of therapeutic response. These lipid nanoparticles may be loaded with functional ligands for targeted delivery and therapeutics. For example, collagen-targeting peptide-conjugated lipid nanoparticles can be used to target exposed collagen type IV, a primary component of the vascular basement membrane exposed in areas of intimal perturbations and pathological vessel wall changes. The addition of radiolabels and fluorescent labels to the lipid nanoparticle can facilitate tracking and enable localization capabilities. For example, collagen-targeting peptide-conjugated lipid nanoparticles with fluorescent labels can be used to co-localize to areas of intimal perturbations, providing a minimally invasive imaging modality for the identification and monitoring of pathological vessel wall changes. Moreover, the lipid nanoparticles can encapsulate biologically active ingredients for targeted therapeutics in a dynamic living environment to elicit effective cellular response. These lipid nanoparticles allow targeted vascular therapeutics, and non-invasive interventions for both the acute and chronic phases of remodeling.

The following description and examples illustrate embodiments of the present disclosure in detail. It is to be understood that this present disclosure is not limited to the particular embodiments described herein and as such can vary. Those of skill in the art will recognize that there are numerous variations and modifications of this present disclosure, which are encompassed within its scope.

Although various features of the present disclosure may be described in the context of a single embodiment, the features may also be provided separately or in any suitable combination. Conversely, although the present disclosure may be described herein in the context of separate embodiments for clarity, the present disclosure may also be implemented in a single embodiment.

The section headings used herein are for organizational purposes only and are not to be construed as limiting the subject matter described.

All terms are intended to be understood as they would be understood by a person skilled in the art. Unless defined otherwise, all technical and scientific terms used herein have the same meaning as commonly understood by one of ordinary skill in the art to which the disclosure pertains.

The following definitions supplement those in the art and are directed to the current application and are not to be imputed to any related or unrelated case, e.g., to any commonly owned patent or application. Although any methods and materials similar or equivalent to those described herein can be used in the practice for testing of the present disclosure, the preferred materials and methods are described herein. Accordingly, the terminology used herein is for the purpose of describing particular embodiments only, and is not intended to be limiting.

### General Definitions

Unless defined otherwise, all technical and scientific terms used herein have the same meaning as commonly understood by one of ordinary skill in the art to which the invention pertains.

All percentages, ratios and proportions herein are by weight, unless otherwise. specified. All temperatures are in degrees Celsius (° C.) unless otherwise specified.

As used herein, “a” and “an” refers to one or to more than one (e.g., to at least one) of the grammatical object of the article. By way of example, “an element” means one element or more than one element.

As used herein, “about” or “approximately” can mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, within 5-fold, or within 2-fold, of a value. Where particular values are described in the application and claims, unless otherwise stated the term “about” meaning within an acceptable error range for the particular value should be assumed.

As used herein, “subject” or “subjects” or “individuals” may include, but are not limited to, mammals such as humans or non-human mammals, e.g., domesticated, agricultural or wild, animals, as well as birds, and aquatic animals. “Patients” are subjects suffering from or at risk of developing a disease, disorder or condition or otherwise in need of the compositions and methods provided herein.

As used herein, “treating” or “treatment” refers to any indicia of success in the treatment or amelioration of the disease or condition. Treating can include, for example, reducing, delaying or alleviating the severity of one or more symptoms of the disease or condition, or it can include reducing the frequency with which symptoms of a disease, defect, disorder, or adverse condition, and the like, are experienced by a patient. As used herein, “treat or prevent” is sometimes used herein to refer to a method that results in some level of treatment or amelioration of the disease or condition, and contemplates a range of results directed to that end, including but not restricted to prevention of the condition entirely.

As used herein, “preventing” refers to the prevention of the disease or condition, e.g., tumor formation, in the patient. For example, if an individual at risk of developing a tumor or other form of cancer is treated with the methods of the present disclosure and does not later develop the tumor or other form of cancer, then the disease has been prevented, at least over a period of time, in that individual.

As used herein, “derivative” as used herein indicates a chemical or biological substance that is related structurally to a second substance and derivable from the second substance through a modification of the second substance. In particular, if a first compound is a derivative of a second compound and the second compound is associated with a chemical and/or biological activity, the first compound differs from the second compound for at least one structural feature, while retaining (at least to a certain extent) the chemical and/or biological activity of the second compound and at least one structural feature (e.g. a sequence, a fragment, a functional group and others) associated thereto. A skilled person will be able to identify, on a case by case basis and upon reading of the present disclosure, structural features of the second compound that have to be maintained in the first compound to retain the second compound chemical and/or biological activity as well as assays that can be used to prove retention of the chemical and/or biological activity. Exemplary “derivatives” can include a prodrug, a metabolite, an enantiomer, a diastereomer, esters (e.g. acyloxyalkyl esters, alkoxycarbonyloxyalkyl esters, alkyl esters, aryl esters, phosphate esters, sulfonate esters, sulfate esters and disulfide containing esters), ethers, amides, carbonates, thiocarbonates, N-acyl derivatives, N-acyloxyalkyl derivatives, quaternary derivatives of tertiary amines, N-Mannich bases, Schiff bases, amino acid conjugates, phosphate esters, metal salts, sulfonate esters, and the like. In some cases, a derivative may include trivial substitutions (i.e., additional alkyl/akylene groups) to a parent compound that retains the chemical and/or biological activity of the parent compound.

As used herein, “administration” or “administering” refers to the introduction of a composition into a subject by a chosen route. For example, if the chosen route is injection, the compositions described herein may be administered by intraperitoneal or intravenous injection. Administration can be effected or performed using any of the various methods and delivery systems known to those skilled in the art. The administering can be performed, for example, but not limited to, intravenously, orally, via implant, transmucosally, transdermally, topically, intramuscularly, intra-articularly, subcutaneously, or extracorporeally. In certain example embodiments, nucleic acid or nucleic acid complexes, such as complexes including nucleic acids and lipids, can be locally or systemically administered to relevant tissues ex vivo, or in vivo through, for example, but not limited thereto, injection, infusion, or stent, with or without their incorporation into biopolymers.

As used herein, a “therapeutically effective amount” refers to the amount of a composition or an active component thereof sufficient to provide a beneficial effect or to otherwise reduce a detrimental non-beneficial event to the individual to whom the composition is administered. By “therapeutically effective dose” herein is meant a dose that produces one or more desired or desirable (e.g., beneficial) effects for which it is administered, such administration occurring one or more times over a given period of time. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); and Pickar, Dosage Calculations (1999)).

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a composition that “comprises,” “has,” “includes” or “contains” one or more elements possesses those one or more elements, but is not limited to possessing only those elements. Likewise, a method that “comprises,” “has,” “includes” or “contains” one or more steps possesses those one or more steps, but is not limited to possessing only those one or more steps.

The term “payload” as used herein refers to compounds enclosed within the lipid nanoparticles. For example, a biological active ingredient is a payload that can be delivered in vivo or in vitro. This term is used interchangeably with the term “active ingredient.”

The term “biologically active ingredient” refers to compounds which when administered to a subject elicits a biological response. Biologically active ingredients include pharmaceutically active ingredients. For example, antipyretics, analgesics, anti-malarials, antibiotics, antiseptics, mood stabilizers, hormone replacements, contraceptives, stimulants, tranquilizers, statins, P-receptor blockers, anti-hypertensives, anticoagulants, bronchodilators, corticosteroids, insulin, and vaccines. Further examples of biologically active ingredients include monoclonal antibodies, immunoglobins, immunosuppressants, interferons, therapeutic antibodies, proteins, enzymes, peptides, DNA and RNA. Further examples of biologically active ingredients include antibiotics, stimulants, statins, b-receptor blockers, anti-hypertensives, anticoagulants, bronchodilators, corticosteroids, insulin, vaccines, immunosuppressants, interferons, antibodies, proteins, and peptides.

As used herein, a “lipid nanoparticle composition” or a “nanoparticle composition” is a composition comprising one or more described lipids. Lipid nanoparticle compositions are typically sized on the order of micrometers or smaller and may include a lipid bilayer. Nanoparticle compositions encompass lipid nanoparticles, liposomes (e.g., lipid vesicles), and lipoplexes. For example, a nanoparticle composition may be a liposome having a lipid bilayer with a diameter of 500 nm or less. The lipid nanoparticles described herein can have a mean diameter of from about 1 nm to about 2500 nm, from about 10 nm to about 1500 nm, from about 20 nm to about 1000 nm, from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, or from about 70 nm to about 80 nm. The lipid nanoparticles described herein can have a mean diameter of about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, 150 nm, or greater. The lipid nanoparticles described herein can be substantially non-toxic.

As used herein, a “PEG lipid” or “PEG-lipid” refers to a lipid comprising a polyethylene glycol component.

As used herein, a “phospholipid” can refer to a lipid that includes a phosphate moiety and one or more carbon chains, such as unsaturated fatty acid chains. A phospholipid may include one or more multiple (e.g., double or triple) bonds. In some embodiments, a phospholipid may facilitate fusion to a membrane. For example, a cationic phospholipid may interact with one or more negatively charged phospholipids of a membrane (e.g., a cellular or intracellular membrane). Fusion of a phospholipid to a membrane may allow one or more elements of a lipid nanoparticle to pass through the membrane, i.e., delivery of the one or more elements to a cell.

The term “therapeutic agent” can refer to any agent that, when administered to a subject, has a therapeutic, diagnostic, and/or prophylactic effect and/or elicits a desired biological and/or pharmacological effect. Therapeutic agents can also be referred to as “actives” or “active agents.” Such agents include, but are not limited to, cytotoxins, radioactive ions, chemotherapeutic agents, small molecule drugs, proteins, and nucleic acids.

The term “nucleic acid” as used herein generally refers to one or more nucleobases, nucleosides, or nucleotides, and the term includes polynucleobases, polynucleosides, and polynucleotides. A nucleic acid can include polynucleotides, mononucleotides, and oligonucleotides. A nucleic acid can include DNA, RNA, or a mixture thereof, and can be single stranded, double stranded, or partially single or double stranded, and can form secondary structures. In some embodiments, a nucleic acid has multiple double-stranded segments and single stranded segments. For example, a nucleic acid may comprise a polynucleotide, e.g., a mRNA, with multiple double stranded segments within it. DNA may be in the form of, e.g., antisense molecules, plasmid DNA, pre-condensed DNA, a PCR product, vectors, expression cassettes, chimeric sequences, chromosomal DNA, or derivatives and combinations of these groups. RNA may be in the form of siRNA, asymmetrical interfering RNA (aiRNA), microRNA (miRNA), mRNA, tRNA, rRNA, tRNA, viral RNA (vRNA), CRISPR RNA, base editor RNA and combinations thereof. Nucleic acids include nucleic acids containing known nucleotide analogs or modified backbone residues or linkages, which are synthetic, naturally occurring, and non-naturally occurring, and which have similar binding properties as the reference nucleic acid. Examples of such analogs include, without limitation, phosphorothioates, phosphoramidates, methyl phosphonates, chiral-methyl phosphonates, 2′, 3′, 4′ and 5′ substituted ribonucleotide, 2′, 3′, 4′ and 5′ substituted 2′-ribonucleotide, substituted and unsubstituted carbocyclic nucleotides, substituted and unsubstituted acyclic nucleotides and peptide-nucleic acids (PNAs). Examples of nucleic acids also include acyclic and carbocyclic nucleotide, such as Glycol nucleic acid. Unless specifically limited, the term encompasses nucleic acids containing known analogues of natural nucleotides that have similar binding properties as the reference nucleic acid. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res., 19:5081 (1991); Ohtsuka etal., J Biol. Chem., 260:2605-2608 (1985); Rossolini et al., Mal. Cell. Probes, 8:91-98 (1994)). “Nucleotides” contain a substituted and/or unsubstituted sugar deoxyribose (DNA), or a substituted and/or unsubstituted sugar ribose (RNA), or a substituted and/or unsubstituted carbocyclic, or a substituted and/or unsubstituted acyclic moiety (e.g., glycol nucleic), a base, and a phosphate group. Nucleotides are linked together through the phosphate groups. “Bases” include purines and pyrimidines, which further include natural compounds adenine, thymine, guanine, cytosine, uracil, inosine, and natural analogs, and synthetic derivatives of purines and pyrimidines, which include, but are not limited to, modifications which place new reactive groups such as, but not limited to, amines, alcohols, thiols, carboxylates, and alkylhalides. The term “gene” refers to a nucleic acid (e.g., DNA or RNA) sequence that comprises partial length or entire length coding sequences necessary for the production of a polypeptide or precursor polypeptide.

Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res., 19:5081 (1991); Ohtsuka et al., J. Biol. Chem., 260:2605-2608 (1985); Rossolini et al., Mol. Cell. Probes, 8:91-98 (1994)).

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

### Multi-Functional Lipid Nanoparticles

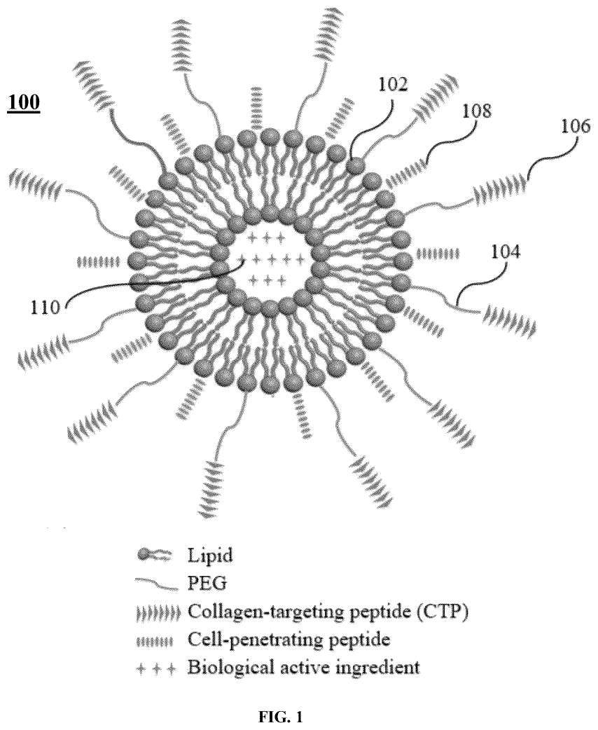

FIG. 1 is an illustrative representation of a multi-functional lipid nanoparticle encapsulated with a biologically active ingredient. The multi-functional lipid nanoparticle 100 has a substantially spherical nanostructure with one or more lipid layers. The lipid layer may include a lipid 102, a pegylated lipid (PEG lipid), a structural lipid or a combination thereof. Polyethylene glycol (PEG) 104 on the lipid layer can be used to reduce protein adsorption and hepatic clearance, thus, enhance the stability of lipid nanoparticles as well as the stability of biologically active ingredient encapsulated therein. PEG 104 also provides an outer surface scaffold for the conjugation of functional ligands. Thus, PEGylated lipid nanoparticles can be effectively functionalized with ligands for e.g., enhanced cellular uptake, cell-specific targeting, triggered release, imaging, and tissue localization.

In some embodiments, multi-functional lipid nanoparticles described herein comprise a PEG lipid. The pegylated lipid (PEG lipid) may include, but are not limited to, PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, the one or more PEG lipids can comprise PEG-dilauroylglycerol, PEG-dimyristoylglycerol (PEG-DMG), PEG-dipalmitoylglycerol, PEG-distearoylglycero-phosphoethanolamine (PEG-DSPE), PEG-dipalmitoylglycerol, PEG-disterylglycerol, PEG-dilaurylglycamide, PEG-dimyristylglycamide, PEG-dipalmitoylglycamide, PEG-disterylglycamide, PEG-cholesterol, and PEG-DMB (3,4-Ditetradecoxylbenzyl-[omega]-methyl-poly(ethylene glycol) ether), 1,2-dimyristoyl-sn-glycero-3-phosphoethanolamine-N-[methoxy(polyethylene glycol)-2000]), PEG-DOPC, or a mixture thereof. In some embodiments, PEG moiety is an optionally substituted linear or branched polymer of ethylene glycol or ethylene oxide. In some embodiments, the PEG moiety is substituted, e.g., by one or more alkyl, alkoxy, acyl, hydroxy, or aryl groups. In some embodiments, the PEG moiety includes PEG copolymer such as PEG-polyurethane or PEG-polypropylene (see, e.g., j. Milton Harris, Poly(ethylene glycol) chemistry: biotechnical and biomedical applications (1992)). In some embodiments, the PEG moiety does not include PEG copolymers, e.g., it may be a PEG monopolymer. Exemplary PEG lipids include, but are not limited to, PEG-dilauroylglycerol, PEG-dimyristoylglycerol (PEG-DMG), PEG-dipalmitoylglycerol, PEG-distearoylglycero-phosphoethanolamine (PEG-DSPE), PEG-dipalmitoylglycerol, PEG-disterylglycerol, PEG-dilaurylglycamide, PEG-dimyristylglycamide, PEG-dipalmitoylglycamide, PEG-disterylglycamide, PEG-cholesterol, and PEG-DMB (3,4-Ditetradecoxylbenzyl-[omega]-methyl-poly(ethylene glycol) ether), 1,2-dimyristoyl-sn-glycero-3-phosphoethanolamine-N-[methoxy(polyethylene glycol)-2000]). In some specific embodiments, the PEG lipid is 1,2-distearoyl-sn-glycero-3-phosphoethanolamine-N-[methoxy(polyethyleneglycol)-2000](DSPE-PEG(2000)).

A PEG lipid may comprise one or more ethylene glycol units, for example, at least 1, at least 2, at least 5, at least 10, at least 20, at least 30, at least 40, at least 50, at least 60, at least 70, at least 80, at least 90, at least 100, at least 120, or at least 150 ethylene glycol units.

In some embodiments, a number average molecular weight of the PEG lipids is from about 200 Da to about 5000 Da. In some embodiments, a number average molecular weight of the PEG lipids is from about 500 Da to about 3000 Da. In some embodiments, a number average molecular weight of the PEG lipids is from about 750 Da to about 2500 Da. In some embodiments, a number average molecular weight of the PEG lipids is from about 750 Da to about 2500 Da. In some embodiments, a number average molecular weight of the PEG lipids is about 500 Da, about 750 Da, about 1000 Da, about 1250 Da, about 1500 Da, about 1750 Da, or about 2000 Da.

In some embodiments, the PEG lipid comprises from about 0.1 mol % to about 15 mol % of the total lipid present in the lipid nanoparticle. In some embodiments, the PEG lipid comprises from about 0.1 mol % to about 20 mol % of the total lipid present in the particle. In some embodiments, the PEG lipid comprises from about 0.5 mol % to about 20 mol % of the total lipid present in the particle. In some embodiments, the PEG lipid comprises from about 1 mol % to about 20 mol % of the total lipid present in the particle. In some embodiments, the PEG lipid comprises from about 0.5 mol % to about 5 mol % of the total lipid present in the particle. In some embodiments, the PEG lipid comprises from about 0.5 mol % to about 2.5 mol % of the total lipid present in the particle.

In some embodiments, multi-functional lipid nanoparticles described herein comprise a helper lipid. “Helper lipids” can refer to lipids that enhance transfection (e.g., transfection of the nanoparticle including the biologically active agent). The mechanism by which the helper lipids enhance transfection includes enhancing particle stability. The helper lipids may include a phospholipid. In some embodiments, the phospholipid comprises a lipid selected from the group consisting of: phosphatidylcholine (PC), phosphatidylethanolamine amine, glycerophospholipid, sphingophospholipids, Guriserohosuhono, sphingolipids phosphono lipids, natural lecithins, and hydrogenated phospholipid. In some embodiments, the phospholipid comprises a phosphatidylcholine. Exemplary phosphatidylcholines include, but are not limited to, soybean phosphatidylcholine, egg yolk phosphatidylcholine (EPC), distearoylphosphatidylcholine, 1,2-distearoyl-sn-glycero-3-phosphocholine (DSPC), dipalmitoyl phosphatidylcholine, dipalmitoyl-sn-glycero-3-phosphocholine (DPPC), palmitoyl oleoyl phosphatidylcholine (POPC), dimyristoyl phosphatidylcholine (DMPC), and dioleoyl phosphatidylcholine (DOPC). In some embodiment, the multi-functional lipid nanoparticles described herein comprise two or more helper lipids.

In some embodiments, the helper lipid is 1,2-dioleoyl-sn-glycero-3-phosphocholine (DOPC), 1,2-distearoyl-sn-glycero-3-phosphoethanolamine (DSPE), 1,2-distearoyl-sn-glycero-3-phosphocholine (DSPC), 1,2-dipalmitoyl-sn-glycero-3-phosphocholine (DPPC), 1,2-dimyristoyl-sn-glycero-phosphocholine (DMPC), 2-oleoyl-1-palmitoyl-sn-glycero-3-phosphocholine (POPC), 1,2-dioleoyl-sn-glycero-3-phosphoethanolamine (DOPE), sphingomyelin, phosphatidyl inositol (PI), or phosphatidic acid (PA). In some specific embodiments, the helper lipid is DOPC.

In some embodiments, the phospholipid comprises a phosphatidylethanolamine amine. In some embodiments, the phosphatidylethanolamine amine is distearoyl phosphatidylethanolamine (DSPE), dipalmitoyl phosphatidyl ethanolamine (DPPE), dioleoyl phosphatidylethanolamine (DOPE), dimyristoyl phosphoethanolamine (DMPE), 16-0-Monome Le PE, 16-0-dimethyl PE, 18-1-trans PE, palmitoyl oleoyl-phosphatidylethanolamine (POPE), or 1-stearoyl-2-oleoyl-phosphatidyl ethanolamine (SOPE). In some embodiments, the phospholipid comprises a glycerophospholipid. In some embodiments, the glycerophospholipid is plasmalogen, phosphatidate, or phosphatidylcholine. In some embodiments, the glycerophospholipid is phosphatidylserine, phosphatidic acid, phosphatidylglycerol, phosphatidylinositol, palmitoyl oleoyl phosphatidylglycerol (POPG), or lysophosphatidylcholine. In some embodiments, the phospholipid comprises a sphingophospholipid. In some embodiments, the sphingophospholipid is sphingomyelin, ceramide phosphoethanolamine, ceramide phosphoglycerol, or ceramide phosphoglycerophosphoric acid. In some embodiments, the phospholipid comprises a natural lecithin. In some embodiments, the natural lecithin is egg yolk lecithin or soybean lecithin. In some embodiments, the phospholipid comprises a hydrogenated phospholipid. In some embodiments, the hydrogenated phospholipid is hydrogenated soybean phosphatidylcholine. In some embodiments, the phospholipid is selected from the group consisting of: phosphatidylcholine, phosphatidylethanolamine, phosphatidylserine, phosphatidylinositol, phosphatidic acid, palmitoyloleoyl phosphatidylcholine, lysophosphatidylcholine, lysophosphatidylethanolamine, dipalmitoylphosphatidylcholine, dioleoylphosphatidylcholine, distearoylphosphatidylcholine, and dilinoleoylphosphatidylcholine.

In some embodiments, the multi-functional lipid nanoparticles comprise a plurality of phospholipids, for example, at least 2, 3, 4, 5, or more distinct phospholipids. In some embodiments, the phospholipid comprises a lipid selected from: 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), and sphingomyelin.

In some embodiments, the helper lipid is a neural lipid. As used herein, the “neutral lipids” suitable for use in a lipid composition of the disclosure include, for example, a variety of neutral, uncharged or zwitterionic lipids. Neutral lipids can function to stabilize and improve processing of the nanoparticles. Examples of neutral lipids suitable for use in the present disclosure include, but are not limited to, 5-heptadecylbenzene-1,3-diol (resorcinol), dipalmitoylphosphatidylcholine (DPPC), distearoylphosphatidylcholine (DSPC), pohsphocholine (DOPC), dimyristoylphosphatidylcholine (DMPC), phosphatidylcholine (PLPC), 1,2-distearoyl-sn-glycero-3-phosphocholine (DAPC), phosphatidylethanolamine (PE), egg phosphatidylcholine (EPC), dilauryloylphosphatidylcholine (DLPC), dimyristoylphosphatidylcholine (DMPC), 1-myristoyl-2-palmitoyl phosphatidylcholine (MPPC), 1-palmitoyl-2-myristoyl phosphatidylcholine (PMPC), 1-palmitoyl-2-stearoyl phosphatidylcholine (PSPC), 1,2-diarachidoyl-sn-glycero-3-phosphocholine (DBPC), 1-stearoyl-2-palmitoyl phosphatidylcholine (SPPC), 1,2-dieicosenoyl-sn-glycero-3-phosphocholine (DEPC), palmitoyloleoyl phosphatidylcholine (POPC), lysophosphatidyl choline, dioleoyl phosphatidylethanolamine (DOPE), dilinoleoylphosphatidylcholine distearoylphosphatidylethanolamine (DSPE), dimyristoyl phosphatidylethanolamine (DMPE), dipalmitoyl phosphatidylethanolamine (DPPE), palmitoyloleoyl phosphatidylethanolamine (POPE), lysophosphatidylethanolamine and combinations thereof.

In some embodiments, the neutral phospholipid may be selected from the group consisting of SPC and dimyristoyl phosphatidyl ethanolamine (DMPE).

In some embodiments, the helper lipid comprises from about 10 mol % to about 90 mol % of the total lipid present in the lipid nanoparticle. In some embodiments, helper lipid comprises from about 10 mol % to about 80 mol % of the total lipid present in the lipid nanoparticle. In some embodiments, helper lipid comprises from about 10 mol % to about 70 mol % of the total lipid present in the lipid nanoparticle. In some embodiments, helper lipid comprises from about 5 mol % to about 50 mol % of the total lipid present in the lipid nanoparticle. In some embodiments, helper lipid comprises from about 5 mol % to about 25 mol % of the total lipid present in the lipid nanoparticle. In some embodiments, helper lipid comprises from about 5 mol % to about 15 mol % of the total lipid present in the lipid nanoparticle. In some embodiments, helper lipid comprises from about 10 mol % to about 25 mol % of the total lipid present in the lipid nanoparticle.

In some embodiments, multi-functional lipid nanoparticles described herein comprise a structural lipid. In some embodiments, the structural lipid comprises a cholesterol or a derivative thereof. The structural lipid can be selected from steroid, sterol, alkyl resoreinol, cholesterol or derivative thereof, fecosterol, sitosterol, ergosterol, campesterol, stigmasterol, brassicasterol, tomatidine, tomatine, ursolic acid, alpha-tocopherol, and a combination thereof. In some embodiments, the structural lipid is a corticosteroid such as prednisolone, dexamethasone, prednisone, and hydrocortisone. In some embodiments, the cholesterol or derivative thereof is cholesterol, 5-heptadecylresorcinol, or cholesterol hemisuccinate. In some embodiments, the cholesterol or derivative thereof is cholesterol.

In some embodiments, the cholesterol or derivative thereof is a cholesterol derivative. In some embodiments, the cholesterol derivative is a polar cholesterol analogue. In some embodiments, the polar cholesterol analogue is 5α-cholestanol, 5β-coprostanol, cholesteryl-(2′-hydroxy)-ethyl ether, cholesteryl-(4′-hydroxy)-butyl ether, or 6-ketocholestanol. In some embodiments, the polar cholesterol analogue is cholesteryl-(4′-hydroxy)-butyl ether. In some embodiments, the cholesterol derivative is a non-polar cholesterol analogue. In some embodiments, the non-polar cholesterol analogue is 5α-cholestane, cholestenone, 5α-cholestanone, 5β-cholestanone, or cholesteryl decanoate.

In some embodiments, the structural lipid comprises from 5 mol % to 70 mol % of the total lipid present in the lipid nanoparticle. In some embodiments, the structural lipid comprises from 5 mol % to 40 mol % of the total lipid present in the lipid nanoparticle. In some embodiments, the structural lipid comprises from 5 mol % to 30 mol % of the total lipid present in the lipid nanoparticle. In some embodiments, the structural lipid comprises from 30 mol % to 60 mol % of the total lipid present in the lipid nanoparticle. In some embodiments, the structural lipid comprises from 35 mol % to 55 mol % of the total lipid present in the lipid nanoparticle. In some embodiments, the cholesterol or the derivative thereof comprises from 5 mol % to 50 mol % of the total lipid present in the lipid nanoparticle. In some embodiments, the cholesterol or the derivative thereof comprises from 5 mol % to 40 mol % of the total lipid present in the lipid nanoparticle. In some embodiments, the cholesterol or the derivative thereof comprises from 5 mol % to 30 mol % of the total lipid present in the lipid nanoparticle.

The lipid nanoparticles can be functionalized with peptide fragments. In some embodiments, the lipid nanoparticle 100 includes a cell-penetrating peptide 108. While PEG improves the stability of lipid nanoparticles, PEGylation also brings negative effects to the delivery of lipid nanoparticles such as low cellular uptake, poor endosomal escape and accelerated blood clearance. The cell-penetrating peptide 108 can facilitate the delivery of lipid nanoparticles and effectively mitigate these the negative effects of PEGylation. The cell-penetrating peptide can be polycationic, amphipathic or hydrophobic. The cell-penetrating peptide can be protein-derived, synthetic or chimeric. Some non-limiting cell-penetrating peptide examples include derivatives of arginine comprising from 4-20 arginine residues (e.g., RRRRRRRR (SEQ TD NO: 6)); protamine and derivatives thereof, for example, protamine sulfate, protamine phosphoric acid, hydrochloric protamine and the like; poly-lysine and poly-histidine comprising 4-20 residues and derivatives thereof, penetratin and derivatives thereof; MPG peptide (GALFLGFLGAAGSTMGAWSQPKSKRKV (SEQ TD NO: 1)) and derivatives thereof, Pep-i peptide (KETWWETWWTEWSQPKKKRKV (SEQ ID NO: 2)) and derivatives thereof; CADY peptide (GLWRALWRLLRSLWRLLWRA (SEQ ID NO: 3)); KALA peptide (pH dependent) (WEAKLAKALAKALAKTILAKALAKALKACEA (SEQ TD NO: 4)); HA2 peptide (GLFGAIAGFIENGWEGMIDG (SEQ TD NO: 5)); histones; polyplexes; polyethyleneimine, polydimethylaminoethyhnethacrylate; polyamidoamine; and poly-R-amino acid esters.

| MPG | Gly-Ala-Leu-Phe-Leu-Gly-Phe-Leu-Gly-Ala-Ala- | SEQ ID NO: 1 |
|---|---|---|
| | Gly-Ser-Thr-Met-Gly-Ala-Trp-Ser-Gln-Pro-Lys- | |
| | Ser-Lys-Arg-Lys-Val | |
| Pep-1 | Lys-Glu-Thr-Trp-Trp-Glu-Thr-Trp-Trp-Thr-Glu- | SEQ ID NO: 2 |
| | Trp-Ser-Gln-Pro-Lys-Lys-Lys-Arg-Lys-Val | |
| CADY peptide | Gly-Leu-Trp-Arg-Ala-Leu-Trp-Arg-Leu-Leu-Arg- | SEQ ID NO: 3 |
| | Ser-Leu-Trp-Arg-Leu-Leu-Trp-Arg-Ala | |
| KALA peptide | Trp-Glu-Ala-Lys-Leu-Ala-Lys-Ala-Leu-Ala-Lys- | SEQ ID NO: 4 |
| | Ala-Leu-Ala-Lys-His-Leu-Ala-Lys-Ala-Leu-Ala- | |
| | Lys-Ala-Leu-Lys-Ala-Cys-Glu-Ala | |
| HA2 | Gly-Leu-Phe-Gly-Ala-Ile-Ala-Gly-Phe-Ile-Glu- | SEQ ID NO: 5 |
| | Asn-Gly-Trp-Glu-Gly-Met-Ile-Asp-Gly | |
| R8 | Arg-Arg-Arg-Arg-Arg-Arg-Arg-Arg | SEQ ID NO: 6 |
| Collagen-targeting | Lys-Leu-Trp-Val-Leu-Pro-Lys | SEQ ID NO: 7 |
| peptide | | |
| Collagen-targeting | Lys-Leu-Trp-Val-Leu-Pro-Lys-Lys | SEQ ID NO: 8 |
| peptide | | |
| Collagen-targeting | Arg-Arg-Ala-Asn-Ala-Ala-Leu-Lys-Ala-Gly-Glu- | SEQ ID NO: 9 |
| peptide | Leu-Tyr-Lys-Cys-Ile-Leu-Tyr | |
| Collagen-targeting | Thr-Lys-Lys-Thr-Leu-Arg-Thr | SEQ ID NO: |
| peptide | | 10 |
| Collagen-targeting | Trp-Arg-Glu-Pro-Ser-Phe-Met-Ala-Leu-Ser | SEQ ID NO: |
| peptide | | 11 |
| Collagen-targeting | Lys-Ile-Trp-Lys-Leu-Pro-Gln | SEQ ID NO: |
| peptide | | 12 |
| Collagen-targeting | Lys-Ile-Phe-Val-Trp-Pro-Tyr | SEQ ID NO: |
| peptide | | 13 |
| Collagen-targeting | Lys-Val-Trp-Ser-Leu-Pro-Gln | SEQ ID NO: |
| peptide | | 14 |
| Collagen-targeting | Arg-Arg-Ala-Asn-Ala-Ala-Leu-Lys-Ala-Gly-Glu- | SEQ ID NO: |
| peptide | Leu-Tyr-Lys-Ser-Ile-Leu-Tyr-Gly-Cys | 15 |

In some embodiments, a cell-penetrating peptide described herein is conjugated with a lipid to form a cell-penetrating lipid. The cell-penetrating peptide can be conjugated with any lipid described herein to form the cell-penetrating lipid. For example, in some embodiments, the cell-penetrating peptide is conjugated with a helper lipid, a PEG lipid, a structural lipid, or a combination thereof to form cell-penetrating lipid(s). The cell-penetrating peptide may be conjugated with a PEG lipid to form the cell-penetrating lipid. For example, the cell-penetrating peptide may be conjugated to pegylated lipids, for example, PEG-distearoylglycero-phosphoethanolamine (PEG-DSPE). In some embodiments, the cell-penetrating peptide is conjugated with a hydrophobic moiety to form the cell-penetrating lipid. The hydrophobic moiety may be a fatty acid, fatty alcohol, or fatty ester. In some embodiments, the hydrophobic moiety is a C8-C30 fatty acid. In some embodiments, the hydrophobic moiety is a C12-C28 fatty acid. In some embodiments, the hydrophobic moiety is a C12-C20 fatty acid. In some embodiments, the hydrophobic moiety is a C15-C24 fatty acid. In some embodiments, the hydrophobic moiety is a C16-C20 fatty acid. In some embodiments, the hydrophobic moiety is a C1O, C11, C12, C13, C14, C15, C16, C17, C18, C19, or C20 fatty acid. In some embodiments, the hydrophobic moiety is a C8-C30 fatty alcohol or fatty ester. In some embodiments, the hydrophobic moiety is a C12-C28 fatty alcohol or fatty ester. In some embodiments, the hydrophobic moiety is a C12-C20 fatty alcohol or fatty ester. In some embodiments, the hydrophobic moiety is a C15-C24 fatty alcohol or fatty ester. In some embodiments, the hydrophobic moiety is a C16-C20 fatty alcohol or fatty ester. In some embodiments, the hydrophobic moiety is a C10, C11, C12, C13, C14, C15, C16, C17, C18, C19, or C20 fatty alcohol or fatty ester. In some embodiments, the cell-penetrating peptide may be covalently attached to a stearic acid to form the cell-penetrating lipid. When the cell-penetrating peptide is octaarginine (R8), it may be covalently attached to a stearic acid to form the cell-penetrating lipid stearic acid-octaarginine (STR-R8). In some embodiments, STR-R8 comprises octaarginine covalently attached to a stearic acid via an amide bond. In some embodiments, STR-R8 has a structure of:

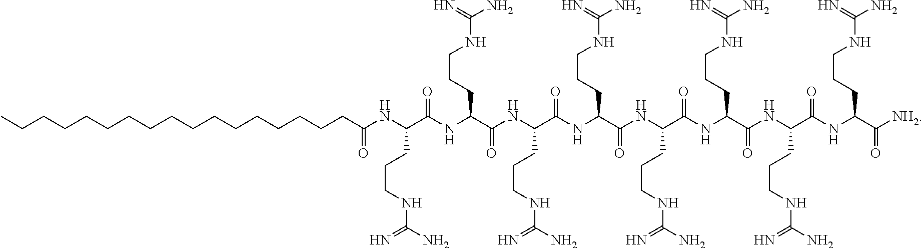 [SMILES: CCCCCCCCCCCCCCCCCC(=O)N[C@@H](CCCNC(=N)N)C(=O)N[C@@H](CCCNC(=N)N)C(=O)N[C@@H](CCCNC(=N)N)C(=O)N[C@@H](CCCNC(=N)N)C(=O)N[C@@H](CCCNC(=N)N)C(=O)N[C@@H](CCCNC(=N)N)C(=O)N[C@@H](CCCNC(=N)N)C(=O)N[C@@H](CCCNC(=N)N)C(N)=O]

In some embodiments, the cell-penetrating peptide comprises from 0.5 mol % to 15 mol % of the total lipid present in the lipid nanoparticle. In some embodiments, the cell-penetrating peptide comprises from about 1 mol % to about 10 mol % of the total lipid present in the lipid nanoparticle. In some embodiments, the cell-penetrating peptide comprises 1 mol %, 1.5 mol %, 2 mol %, 2.5 mol %, 3 mol %, 3.5 mol %, 4 mol %, 4.5 mol %, 5 mol %, 5.5 mol %, 6 mol %, 6.5 mol %, 7 mol %, 4.5 mol %, 5 mol %, 5.5 mol %, 6 mol %, 6.5 mol %, 7 mol %, 7.5 mol %, 8 mol %, 8.5 mol %, 9 mol %, 9.5 mol % or 10 mol % of the total lipid present in the particle. In some embodiments, the cell-penetrating lipid comprises from 0.5 mol % to 15 mol % of the total lipid present in the lipid nanoparticle. In some embodiments, the cell-penetrating lipid comprises from about 1 mol % to about 80 mol % of the total lipid present in the lipid nanoparticle. In some embodiments, the cell-penetrating lipid comprises from about 50 mol % to about 80 mol % of the total lipid present in the lipid nanoparticle. In some embodiments, the cell-penetrating lipid comprises from about 1 mol % to about 10 mol % of the total lipid present in the lipid nanoparticle. In some embodiments, the cell-penetrating lipid comprises from about 5 mol % to about 10 mol % of the total lipid present in the lipid nanoparticle. In some embodiments, the cell-penetrating lipid comprises 1 mol %, 1.5 mol %, 2 mol %, 2.5 mol %, 3 mol %, 3.5 mol %, 4 mol %, 4.5 mol %, 5 mol %, 5.5 mol %, 6 mol %, 6.5 mol %, 7 mol %, 4.5 mol %, 5 mol %, 5.5 mol %, 6 mol %, 6.5 mol %, 7 mol %, 7.5 mol %, 8 mol %, 8.5 mol %, 9 mol %, 9.5 mol % or 10 mol % of the total lipid present in the particle.

In some embodiments, a multi-functional lipid nanoparticle described herein comprises a collagen-targeting peptide (CTP). In some embodiments, the multi-functional lipid nanoparticle 100 comprises a collagen-targeting peptide (CTP) 106. The collagen-targeting peptide may be conjugated to the lipid layer. As illustrated in FIG. 1, the collagen-targeting peptide may be conjugated to the surface (e.g., exterior surface) of the lipid layer. In some embodiments, the collagen-targeting peptide is conjugated with a lipid to form a collagen-targeting lipid. The collagen-targeting peptide can be conjugated with any lipid described herein to form the collagen-targeting lipid. For example, in some embodiments, the collagen-targeting peptide is conjugated with a helper lipid, a PEG lipid, a structural lipid, or a combination thereof to form collagen-targeting lipid(s). In other embodiments, the collagen-targeting peptide is conjugated to a PEG lipid to form a collagen-targeting lipid. In some embodiment, the collagen-targeting lipid comprises pegylated 1,2-distearoyl-sn-glycero-3-phosphoethanolamine (DSPE).

In some embodiments, the collagen-targeting peptide comprises a collagen IV heptapeptide that preferentially binds to collagen type IV, an abundant sub-endothelial matrix protein exposed during vascular intervention and primary component of the vascular basement membrane exposed in areas of endothelial perturbations and pathological vessel walls. In some specific embodiments, the collagen-targeting peptide comprises a sequence of KLWVLPK (SEQ ID NO. 7). The collagen-targeting peptide may comprise one or more modification, for example, with an azido-modified lysine (SEQ ID NO. 8). CTP-conjugated lipid nanoparticles can be targeted to exposed collagen matrices specific to areas of vascular pathology and enhance targeted vascular uptake/delivery. In some embodiments, the collagen-targeting peptide comprises a peptide derived from the platelet receptor that binds to al chains in collagen (SEQ ID NO: 9), a peptide derived from the collagen binding domains in the collagen-degrading enzyme, collagenase (SEQ ID NO: 10), or a peptide derived from the von Willeband's factor (vWF), which is an adhesive glycoprotein found in plasma, platelets, and endothelial cells (SEQ ID NO: 11). In some embodiments, the collagen-targeting peptide comprises a sequence of KIWKLPQ (SEQ ID NO: 12), KIFVWPY (SEQ ID NO: 13), KVWSLPQ (SEQ ID NO: 14), RRANAALKAGELYKSILYGC (SEQ ID NO: 15).

In some embodiments, the collagen-targeting peptide comprises from 0.5 mol % to 15 mol % of the total lipid present in the particle. In some embodiments, the collagen-targeting peptide comprises from about 1 mol % to about 10 mol % of the total lipid present in the particle. In some embodiments, the collagen-targeting peptide comprises 1 mol %, 1.5 mol %, 2 mol %, 2.5 mol %, 3 mol %, 3.5 mol %, 4 mol %, 4.5 mol %, 5 mol %, 5.5 mol %, 6 mol %, 6.5 mol %, 7 mol %, 4.5 mol %, 5 mol %, 5.5 mol %, 6 mol %, 6.5 mol %, 7 mol %, 7.5 mol %, 8 mol %, 8.5 mol %, 9 mol %, 9.5 mol % or 10 mol % of the total lipid present in the particle. In some embodiments, the collagen-targeting lipid comprises from 0.5 mol % to 30 mol % of the total lipid present in the particle. In some embodiments, the collagen-targeting lipid comprises from 5 mol % to 10 mol % of the total lipid present in the particle. In some embodiments, the collagen-targeting lipid comprises from about 1 mol % to about 10 mol % of the total lipid present in the particle. In some embodiments, the collagen-targeting lipid comprises 1 mol %, 1.5 mol %, 2 mol %, 2.5 mol %, 3 mol %, 3.5 mol %, 4 mol %, 4.5 mol %, 5 mol %, 5.5 mol %, 6 mol %, 6.5 mol %, 7 mol %, 4.5 mol %, 5 mol %, 5.5 mol %, 6 mol %, 6.5 mol %, 7 mol %, 7.5 mol %, 8 mol %, 8.5 mol %, 9 mol %, 9.5 mol % or 10 mol % of the total lipid present in the particle.

In some embodiments, a multi-functional lipid nanoparticle described herein comprises means such as an optical label or a radiolabel to facilitate tracking of the lipid nanoparticles. In some embodiments, the multi-functional lipid nanoparticle 100 comprises optical labels or radiolabels to facilitate tracking of the lipid nanoparticles, allowing efficient imaging-guided personalized diagnosis and treatment. The optical labels may include fluorophores and quantum dots. For example, fluorescent dyes may be inserted into the lipid layer during the formation of the lipid nanoparticles. Alternatively, quantum dots may be conjugated to lipid layers or encapsulated in the lipid nanoparticles.

In some embodiments, a multi-functional lipid nanoparticle described herein comprises a biologically active ingredient as a payload. In some embodiments, the multi-functional lipid nanoparticle 100 encapsulates a biologically active ingredient 110. The lipid nanoparticles described herein can be designed to deliver a payload, such as a therapeutic agent, or a target of interest. Exemplary therapeutic agents include, but are not limited to, antibodies (e.g., monoclonal, chimeric, humanized, nanobodies, and fragments thereof etc.), cholesterol, hormones, peptides, proteins, chemotherapeutics and other types of antineoplastic agents, low molecular weight drugs, vitamins, co-factors, nucleosides, nucleotides, oligonucleotides, enzymatic nucleic acids, antisense nucleic acids, triplex forming oligonucleotides, antisense DNA or RNA compositions, chimeric DNA:RNA compositions, allozymes, aptamers, ribozyme, decoys and analogs thereof, plasmids and other types of expression vectors, and small nucleic acid molecules, RNAi agents, short interfering nucleic acid (siRNA), small activating RNA (saRNA), messenger ribonucleic acid (messenger RNA, mRNA), short interfering RNA (siRNA), double-stranded RNA (dsRNA), micro-RNA (miRNA), and short hairpin RNA (shRNA) molecules, peptide nucleic acid (PNA), a locked nucleic acid ribonucleotide (LNA), morpholino nucleotide, threose nucleic acid (TNA), glycol nucleic acid (GNA), siRNA (small internally segmented interfering RNA), aiRNA (asymmetric interfering RNA), and siRNA with 1, 2 or more mismatches between the sense and anti-sense strand to relevant cells and/or tissues, such as in a cell culture, subject or organism. Therapeutic agents can be purified or partially purified, and can be naturally occurring or synthetic, or chemically modified. In some embodiments, the therapeutic agent is an RNAi agent, short interfering nucleic acid (siNA), small activating RNA (saRNA), short interfering RNA (siRNA), double-stranded RNA (dsRNA), micro-RNA (miRNA), or a short hairpin RNA (shRNA) molecule.

In some embodiments, the payload comprises one or more nucleic acid(s) (i.e., one or more nucleic acid molecular entities). In some embodiments, the nucleic acid is a single-stranded nucleic acid. In some embodiments, single-stranded nucleic acid is a DNA. In some embodiments, single-stranded nucleic acid is an RNA. In some embodiments, the nucleic acid is a double-stranded nucleic acid. In some embodiments, the double-stranded nucleic acid is a DNA. In some embodiments, the double-stranded nucleic acid is an RNA. In some embodiments, the double-stranded nucleic acid is a DNA-RNA hybrid. In some embodiments, the nucleic acid is a messenger RNA (mRNA), a microRNA, an asymmetrical interfering RNA (aiRNA), a small hairpin RNA (shRNA), or a Dicer-Substrate dsRNA.

In some embodiments, the payload comprises therapeutically effective agents that target proteins or genes that are associated with vascular pathogenesis and vascular pathway remodeling, including but not limited to matrix metalloproteinase-2 (MMP2), matrix metalloproteinase-9 (MMP9), matrix metalloproteinase-14 (MMP14), and matrix metalloproteinase-3 (MMP3). For example, the therapeutically effective agents may be siRNA.

The amount of a biologically active ingredient in the lipid nanoparticle may depend on the size, composition, desired target and/or application, or other properties of the nanoparticle composition. For example, the amount of RNA comprised in a nanoparticle composition may depend on the size, sequence, and other characteristics of the RNA. The relative amounts of a therapeutic agent and other elements (e.g., lipids) in a nanoparticle composition may also vary. In some embodiments, the wt/wt ratio (i.e., weight ratio) of the lipid component to a therapeutic agent in a nanoparticle composition may be from about 5:1 to about 150:1, such as about 5:1, 6:1, 7:1, 8:1, 9:1, 10:1, 11:1, 12:1, 13:1, 14:1, 15:1, 16:1, 17:1, 18:1, 19:1, 20:1, 25:1, 30:1, 35:1, 40:1, 45:1, 50:1, 60:1, 70:1, 80:1, 90:1, 100:1, 110:1, 120:1, 130:1 140:1, 150:1. For example, the wt/wt ratio of the lipid component to a therapeutic agent may be from about 20:1 to about 100:1. The amount of a therapeutic agent in a nanoparticle composition can be measured using absorption spectroscopy (e.g., ultraviolet-visible spectroscopy). In some embodiments, the wt/wt ratio of the lipid component to a therapeutic agent (e.g., siRNA) in a nanoparticle composition is about 100:1.

In some embodiments, the wt/wt ratio of the lipid component to a therapeutic agent in a nanoparticle composition is about 90:1 to about 110:1. In some embodiments, the wt/wt ratio of the lipid component to a therapeutic agent in a nanoparticle composition is about 80:1 to about 120:1. In some embodiments, the wt/wt ratio of the lipid component to a therapeutic agent in a nanoparticle composition is about 10:1 to about 150:1. In some embodiments, the wt/wt ratio of the lipid component to a therapeutic agent in a nanoparticle composition is about 50:1 to about 150:1. In some embodiments, the wt/wt ratio of the lipid component to a therapeutic agent in a nanoparticle composition is about 25:1 to about 200:1. In some embodiments, the wt/wt ratio of the lipid component to a therapeutic agent in a nanoparticle composition is at least 20:1, at least 50:1, at least 75:1 at least 100:1, at least 150:1, at least 200:1, at least 300:1, at least 400:1, or at least 500:1. In some embodiments, the wt/wt ratio of the lipid component to a therapeutic agent in a nanoparticle composition is at most 25:1, at most 50:1, at most 75:1 at most 100:1, at most 150:1, at most 200:1, at most 300:1, at most 400:1, at most 500:1, or at most 1000:1. In some embodiments, therapeutic agent is an RNA.

In some embodiments, the wt/wt ratio of total lipids to a nucleic acid in a nanoparticle described herein is about 10:1 to about 150:1. In some embodiments, the wt/wt ratio of total lipids to a nucleic acid in a nanoparticle described herein is at least 10:1, at least 20:1, at least 50:1, at least 75:1 at least 100:1, at least 150:1, at least 200:1, at least 300:1, at least 400:1, or at least 500:1. In some embodiments, the wt/wt ratio of total lipids to a nucleic acid in a nanoparticle described herein is at most 25:1, at most 50:1, at most 75:1 at most 100:1, at most 150:1, at most 200:1, at most 300:1, at most 400:1, at most 500:1, or at most 1000:1. In some embodiments, the wt/wt ratio of total lipids to a nucleic acid in a nanoparticle described herein is about 100:1. In some embodiments, the wt/wt ratio of total lipids to a nucleic acid in a nanoparticle described herein is 90:1 to about 110:1. In some embodiments, the wt/wt ratio of total lipids to a nucleic acid in a nanoparticle described herein is about 80:1 to about 120:1. In some embodiments, the wt/wt/ratio of total lipids to a nucleic acid in a nanoparticle described herein is about 50:1 to about 150:1. In some embodiments, the wt/wt ratio of total lipids to a nucleic acid in a nanoparticle described herein is about 25:1 to about 200:1.

In some embodiments, lipid nanoparticles described herein are formed with an average encapsulation efficiency ranging from about 10% to about 30%, from about 30% to about 50%, from about 50% to about 70%, from about 70% to about 90%, or from about 90% to about 100%. In some embodiments, the lipid nanoparticles are formed with an average encapsulation efficiency ranging from about 75% to about 95%. In some embodiments, a nucleic acid (e.g., RNA) entrapment efficiency of a nanoparticle described herein is at least 10%, at least 20%, at least 30%, at least 40%, at least 50%, at least 60%, at least 70%, at least 80%, at least 90% or at least 95%. In some embodiments, a nucleic acid (e.g., RNA) entrapment efficiency of a nanoparticle described herein is at least 95%. In some embodiments, a nucleic acid (e.g., RNA) entrapment efficiency of a nanoparticle described herein is at least 98%. In some embodiments, a nucleic acid (e.g., RNA) entrapment efficiency of a nanoparticle described herein is at least 99%. In some embodiments, a nucleic acid (e.g., RNA such as siRNA) is almost 100% internalized in the LNP. In some embodiments, a nucleic acid (e.g., RNA such as siRNA) is at least 99% internalized in the LNP. In some embodiments, a nucleic acid (e.g., RNA such as siRNA) is at least 98% internalized in the LNP. In some embodiments, a nucleic acid (e.g., RNA such as siRNA) is at least 95% internalized in the LNP. In some embodiments, a nucleic acid (e.g., RNA such as siRNA) is at least 90% internalized in the LNP.

In some embodiment, multi-functional lipid nanoparticles described herein comprise a nucleic acid condenser. In some embodiment, when nucleic acid is encapsulated as the biologically active ingredient, the multi-functional lipid nanoparticle 100 comprises one or more types of nucleic acid condensers to facilitate the loading and encapsulation of nucleic acid. Nucleic acid condensers may include multivalent metal ions, inorganic cations, polyamines, protamines, peptides, lipids, and liposomes. In some embodiments, multivalent cationic charged ligands are used as nucleic acid condensers to induce the interactions with nucleic acid to be encapsulated. These multivalent cationic charged ligands can be conjugated to lipids in order to drive higher entrapment within lipid layers.

In some embodiments, nucleic acid condensers described herein comprise polyarginine, protamine sulfate, protamine phosphoric acid, hydrochloric protamine, polylysine, polyhistidine, penetratin and derivatives thereof, MPG peptide, Pep-1 peptide, CADY peptide, KALA peptide, HA2 peptide, histones, polyplexes, polyethyelenimine, polydimethylamino-ethylmethacrylate, polyamidoamine, or poly-β-amino acid esters or combinations thereof. For example, R8 (SEQ ID NO: 7 or SEQ ID NO: 8) can be used as nucleic acid condensers. Alternatively or additionally, the nucleic acid condensers may include monovalent cations and divalent cations. Divalent cations may include calcium (“Ca”), magnesium (“Mg”), or ferrous (“Fe”). In some embodiments, the nucleic acid condenser is calcium (“Ca”). The concentrations of the monovalent cations or divalent cations may be in a range of about 5 mM to about 50 mM, about 10 mM to about 40 mM, about 10 mM to about 30 mM, and about 5 mM to about 15 mM.

In some embodiment, multi-functional lipid nanoparticles described herein comprise at least two different nucleic acid condensers. In some embodiments, the lipid nanoparticle 100 includes different types of nucleic acid condensers. For example, the lipid nanoparticles encapsulated with nucleic acid as biologically active ingredient may include R8 (SEQ ID NO: 7 or SEQ ID NO: 8) and divalent cations (e.g., Ca). R8 functions as cell-penetrating peptide and nucleic acid condenser because of its polycationic property and induces electrostatic interactions between nucleic acid segments and multivalent cationic residues to promote the loading of nucleic acid segments. In some embodiments, the nucleic acid condensers comprise a divalent cations (e.g., Ca) and R8 conjugated to a lipid or to a hydrophobic moiety (e.g., STR-R8).

In some embodiments, lipid nanoparticles described herein have a median diameter of about 10 nm to about 500 nm. In some embodiments, the median diameter of the lipid nanoparticles is from about 50 nm to about 150 nm, from about 60 nm to about 140 nm, from about 70 nm to about 130 nm, from about 80 nm to about 120 nm, or from about 90 nm to about 110 nm. In some embodiments, the median diameter of the lipid nanoparticles is about 40 nm, about 50 nm, about 60 nm, about 65 nm, about 70 nm, about 75 nm, about 80 nm, about 85 nm, about 90 nm, about 95 nm, about 100 nm, about 105 nm, about 110 nm, about 115 nm, or about 120 nm. Particle size and particle size distribution of the nanoparticles can be measured by light scattering. In some embodiments, the particle size distribution is unimodal.

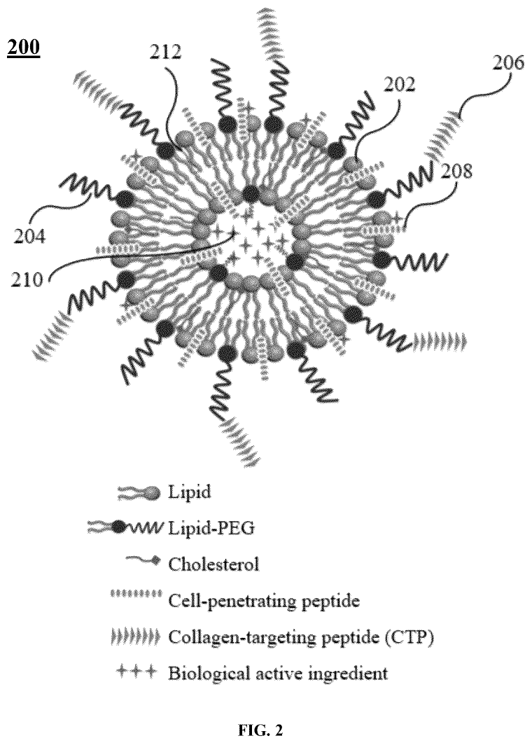

FIG. 2 is an illustrative representation of another multi-functional lipid nanoparticle encapsulated with a biologically active ingredient 210. The multi-functional lipid nanoparticle 200 comprises a lipid layer comprising lipids (e.g., helper lipids) 202, PEG lipids 204 and structural lipids (e.g., cholesterol) 212. In some embodiments, the collagen-targeting peptide (CTP) 206 is conjugated to PEG lipid 204 to form a collagen-targeting lipid, which is inserted into the lipid layer of the nanoparticle 200. The cell-penetrating peptide 208 is conjugated to the lipid layer. Different from the multi-functional lipid nanoparticle 100 where the cell-penetrating peptides 108 are inserted to an exterior surface of the lipid layer, here, the peptides 208 is inserted to both exterior and interior surface of the lipid layer.

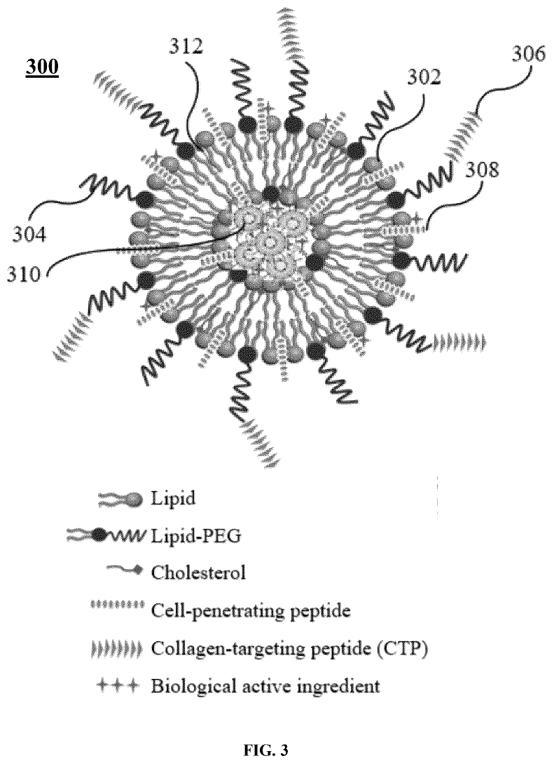

FIG. 3 is an illustrative representation of another multi-functional lipid nanoparticle encapsulated with multi-functional lipid nanoparticles and biologically active ingredient. The multi-functional lipid nanoparticle 300 comprises a lipid layer comprising lipids (e.g., helper lipids) 302, PEG lipids 304 and structural lipids (e.g., cholesterol) 312. In some embodiments, the collagen-targeting peptide (CTP) 306 is conjugated to PEG lipid 304 to form a collagen-targeting lipid which is inserted into the lipid layer of the nanoparticle 300. Different from FIGS. 1 and 2 illustrating a single unit of the lipid nanoparticle, the lipid nanoparticle 300 encapsulates a plurality of units of lipid nanoparticles and biologically active ingredients. In other words, whereas the lipid nanoparticles 100 and 200 have a core with aqueous solution containing biologically active ingredients, the lipid nanoparticle 300 has a dense core containing lipid nanoparticles and biologically active ingredients. The addition of biologically active ingredient can be a defining factor in creating the dense core of the lipid nanoparticle.

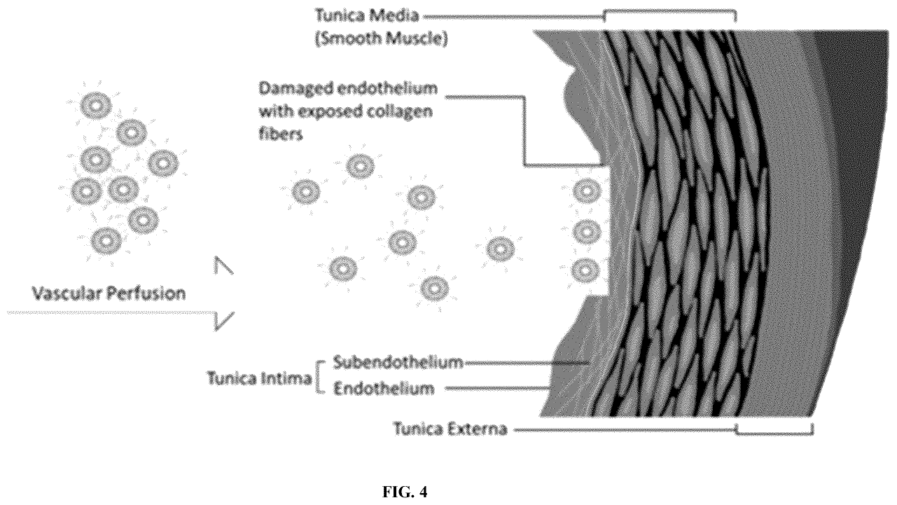

FIG. 4 is an illustrative representation of multi-functional lipid nanoparticles delivered to damaged endothelium with exposed collagen fibers. The lipid nanoparticles as described herein and illustrated in FIGS. 1-3 allow simultaneous diagnosis, targeted drug delivery, and monitoring of therapeutic response. The conjugated collagen-targeting peptides allow targeted binding of lipid nanoparticles to collagen proteins on the damaged endothelium. The cell-penetrating peptides facilitate the delivery of lipid nanoparticles and effectively mitigate the negative effects of PEGylation in the lipid layer. The incorporated imaging labels allow targeted imaging of areas of interest. Additionally, the biologically active ingredients are encapsulated with high efficacy, enabling targeted delivery of therapeutically effective agents in a dynamic living environment to elicit effective cellular response.

In some embodiments, multi-functional lipid nanoparticles are prepared via thin-film hydration assembly and optionally, followed by extrusion. A lipid film is formed by dissolving lipids (e.g., helper lipid, PEG lipid, structural lipid, collagen-targeting peptide-conjugated lipid) in organic solvent in a flask and removing the organic solvent in vacuum. After the solvent is completely removed, the lipid film is hydrated by an aqueous solution, thereby forming lipid nanoparticles.

In some embodiments, multi-functional lipid nanoparticles are prepared via ethanol injection. An ethanolic solution of lipids is rapidly injected into an aqueous solution through a needle, dispersing the lipids throughout the solution and promoting the formation of lipid nanoparticles.

In some embodiments, multi-functional lipid nanoparticles encapsulated with biologically active ingredient are prepared via impingement jet mixing. A syringe pump drives two opposing liquid streams at high velocity into a mixing chamber. One of the streams contains lipids in organic solvent whereas the other stream contains biologically active ingredient. The mixing at high velocity reduces the solubility of the lipids so that homogenous lipid nanoparticles are formed with biologically active ingredient encapsulated therein.

In some embodiments, microfluidic techniques are used to produce multi-functional lipid nanoparticles. Lipids in organic solvent and biologically active ingredient in aqueous solution are intersected, in a controllable manner, into a rapid mixing microfluidic device, where lipid nanoparticles encapsulated with biologically active ingredient are formed. T-junction mixing is another method of rapid mixing operated at a high flow rate where lipid in organic solvent and biologically active ingredient in aqueous solution flow through two inlet channels and are mixed.

In some embodiments, multi-functional lipid nanoparticles encapsulated with biologically active ingredient are prepared via extrusion. Dispersed lipid nanoparticles flow through a filter or a membrane with a uniform pore size distribution, to generate a homogeneous group of smaller lipid nanoparticles. Extrusion process requires to be performed in the presence of the biologically active ingredient to be encapsulated. Large lipid nanoparticles are resealed rapidly, resulting in entrapment of the biologically active ingredient. A syringe with a standard sterilization filter holder can be used to perform the extrusion and membrane homogenization. To further reduce the size of lipid nanoparticles, multiple extrusions through a polycarbonate membrane can be used. Both the number of extrusion cycles and the size of membrane pores may determine the degree of size reduction. The size of membrane pores may be about 2 μm, about 0.5 μm, about 0.2 μm, about 0.1 μm, about 0.05 μm.

The present disclosure is further described in the following Examples which are given for illustration purposes only and are not intended to limit the disclosure in any way. In particular, the Examples below describe the synthesis of collagen-targeting peptide (CTP)-conjugated lipid and assembly of different types of lipid nanoparticles and encapsulation of biologically active ingredients. The assembly of base lipid nanoparticles (PLPs) without functional modification or encapsulation, as well as collagen-targeting peptide-conjugated lipid nanoparticles (CTP-PLPs) and cell-penetrating peptide-conjugated lipid nanoparticles (R8-PLPs) are also described. The acronyms of constituents for lipid nanoparticle assembly are listed in Table 1.

TABLE 1
Lipid Nanoparticle Constituent Acronyms Defined

| Acronym | Lipid Derivative/Constituent |
|---|---|
| DSPE-PEG | 1,2-distearoyl-sn-glycero-3-phosphoethanolamine-N- |
| | [methoxy(polyethylene glycol)-2000] |
| DSPE-PEG- | 1,2-distearoyl-sn-glycero-3-phosphoethanolamine-N- |
| DBCO | [dibenzocyclooctyl(polyethylene glycol)-2000] |
| DOPC | 1,2-dioleoyl-sn-glycero-3-phosphocholine |
| Rho-PE | 1,2-dioleoyl-sn-glycero-3-phosphoethanolamine-N- |
| | (lissamine rhodamine B sulfonyl) |
| Cy7-PE | 1,2-dioleoyl-sn-glycero-3-phosphoethanolamine-N- |
| | (Cyanine 7) |
| Chol | Ovine cholesterol |
| STR-R8 | stearic acid-octaarginine |
| CTP | KLWVLPKK(N3)-NH2 |

### Example 1: Collagen-Targeting Peptide (CTP)-Conjugated Lipid Synthesis

In this example, collagen-targeting peptide-conjugated lipid was synthesized via click chemistry. 1, 2-distearoyl-sn-glycero-3-phosphoethanolamine-N-[dibenzocydooctyl (polyethylene glycol)-2000](DSPE-PEG-DBCO) was a form of 1, 2-distearoyl-sn-glycero-3-phosphoethanolamine-N-[methoxy(polyethylene glycol)-2000](DSPE-PEG). DSPE-PEG-DBCO lipid had a cyclooctyne modification that can be used in azide-alkyne cycloaddition reactions. The CTP used herein had the sequence of KLWVLPKK (SEQ ID NO: 8) and possessed a high binding affinity to collagen type IV proteins. The CTP was equipped with an unnatural-lysine with an azide end-group for click chemistry reaction. Briefly, an equal molar of azido-modified CTP and DSPE-PEG-DBCO lipid were mixed at room temperature under constant agitation overnight to yield the product of collagen-targeting peptide-conjugated lipid DSPE-PEG-CTP. See, Grimsley et al., Pharmaceutics, 13, 1816 (2021), which is incorporated herein by reference.

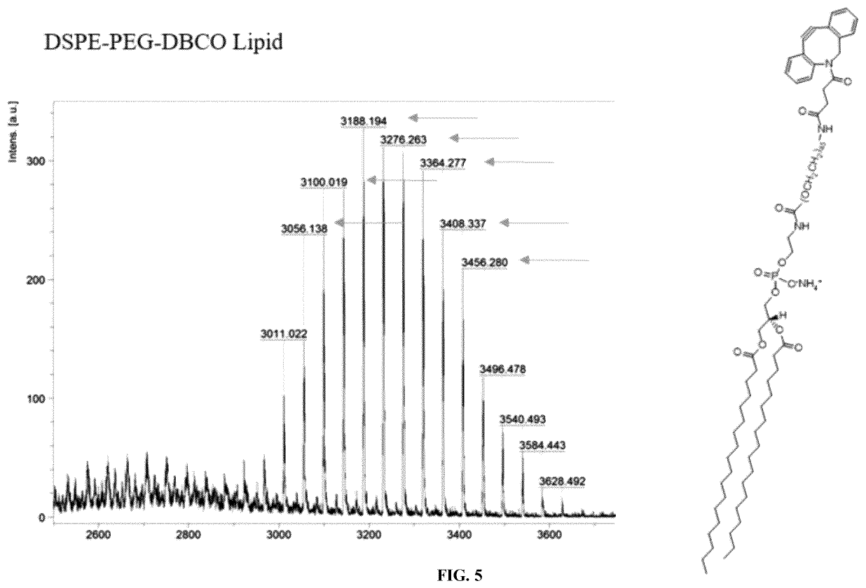

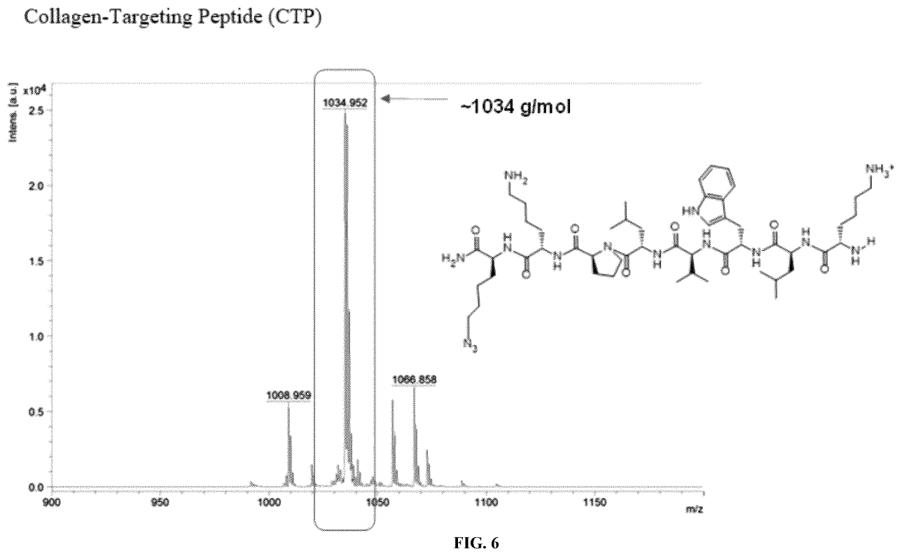

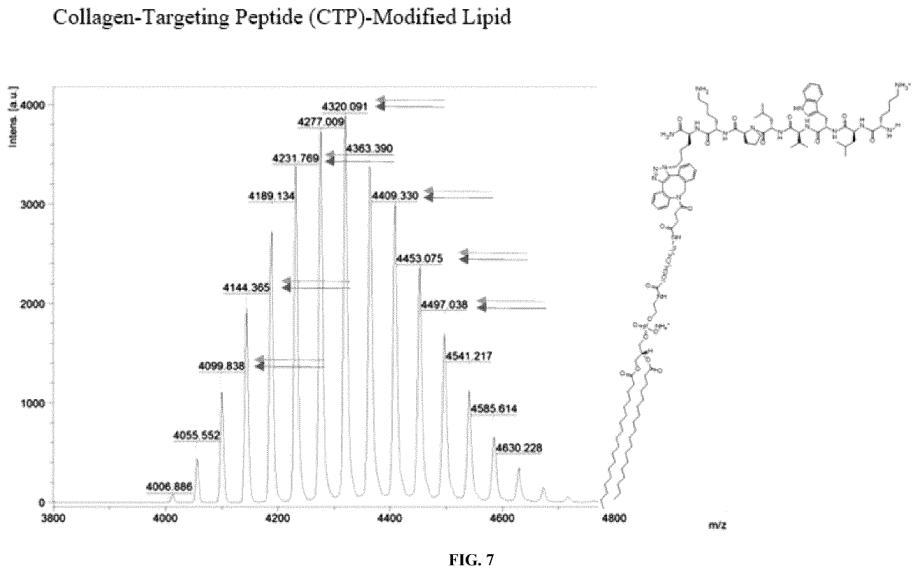

FIGS. 5-7 demonstrate the synthesis of collagen-targeting peptide (CTP)-conjugated lipid. FIG. 5 illustrates the mass spectrum and chemical structure of DSPE-PEG-DBCO lipid with cyclooctyne modification. DSPE-PEG-DBCO lipid has various characteristic peaks as labeled. FIG. 6 illustrates the mass spectrum and chemical structure of an example collagen-targeting peptide (CTP) with an azide end-group. A characteristic peak as labeled is about 1034 m/z. FIG. 7 illustrates the mass spectrum and chemical structure of a collagen-targeting peptide-conjugated lipid, where the CTP and DSPE-PEG-DBCO are conjugated via click chemistry. The peaks of aggregate molecular weight labeled in green arrows are shifted compared to the peaks in the pre-clicked DSPE-PEG-DBCO spectrum, demonstrating the conjugation of the CTP to the PEG-lipid.

Examples 2-6 described the assembly of different types of lipid nanoparticles and encapsulation with biologically active ingredients. Among others, Examples 2-4 described the assembly of base lipid nanoparticles (PLP), collagen-targeting peptide-conjugated lipid nanoparticles (CTP-PLP) and cell-penetrating peptide-conjugated lipid nanoparticles (R8-PLP), respectively. These assembled nanoparticles, namely, PLP, CTP-PLP and R8-PLP, were used as control groups for performance comparison with multi-functional lipid nanoparticles as described herein. Examples 5 and 6 described the assembly of multi-functional lipid nanoparticles conjugated with collagen-targeting peptides and cell-penetrating peptides (CTP-R8-PLP or R8-CTP-PLP interchangeably) and the encapsulation of biologically active ingredients therein, respectively.

### Example 2: Base Lipid Nanoparticle (PLP) Assembly

Base lipid nanoparticles were assembled and used as control groups. Lipids 1,2-dioleoyl-sn-glycero-3-phosphocholine (DOPC) and ovine cholesterol (Chol) were mixed at a mole ratio of 7:3. 10 mol % DSPE-PEG was added into the mixture of DOPC and Chol. Optionally, about 0.1 mol %-about 0.5 mol % Rho-PE or about 0.1 mol %-about 0.5 mol % Cy7-PE was added into the mixture for fluorescent labeling. The mixture was dissolved in CHCl₃ and dried under inert gas (e.g., nitrogen, argon and the like) and vacuum to remove any remaining solvent, in order to form a dry lipid film. The lipid film was subsequently resuspended in 100% molecular grade EtOH and injected dropwise into 10 mM Tris-HCl at pH 8.0 and a 2:3 EtOH:aqueous volume ratio, under constant vortexing at room temperature. For example, 600 ul 10 mM Tris-HCl containing 10 nM CaCl₂ at pH 8.0 was injected with 1000 ug total lipid in 400 uL EtOH, under constant vortexing at room temperature. The formed lipid nanoparticles were then purified from remaining solvent via 24 h dialysis against phosphate-buffered saline (PBS) buffer at pH 7.4 at 4° C. and extruded at 100 nm using polycarbonate membranes prior to characterization.

### Example 3: Collagen-Targeting Peptide-Conjugated Lipid Nanoparticle (CTP-PLP) Assembly

Collagen-targeting peptide-conjugated lipid nanoparticles (CTP-PLP) were formed via pre-insertion. Lipids 1,2-dioleoyl-sn-glycero-3-phosphocholine (DOPC) and ovine cholesterol (Chol) were mixed at a mole ratio of 7:3. Unlike the assembly of base lipid nanoparticles where 10 mol % DSPE-PEG was added into the mixture, here, 0.5 mol %-10 mol % of CTP-conjugated lipid (DSPE-PEG-CTP), as described in Example 1, substitutes an equal mol % of DSPE-PEG and was added into the mixture of DOPC and Chol. With the substitution of the same mol % of DSPE-PEG with DSPE-PEG-CTP, the PEGylation on the lipid layer was maintained at about 10%. Optionally, about 0.1 mol %-about 0.5 mol % Rho-PE or about 0.1 mol %-about 0.5 mol % Cy7-PE was added into the mixture for fluorescent labeling. The mixture was dissolved in CHCl₃ and dried under inert gas (e.g., nitrogen, argon and the like) and vacuum to remove any remaining solvent, in order to form a dry lipid form. The lipid film was then resuspended in 100% molecular grade EtOH and injected dropwise into 10 mM Tris-HCl at pH 8.0 and a 2:3 EtOH:aqueous volume ratio, under constant vortexing at room temperature. For example, 600 ul 10 mM Tris-HCl containing 10 nM CaCl₂) at pH 8.0 was injected with 1000ug total lipid in 400 uL EtOH, under constant vortexing at room temperature. The formed CTP-PLPs were then purified from remaining solvent via 24 h dialysis against phosphate-buffered saline (PBS) buffer at pH 7.4 at 4° C. and extruded. As such, the CTP-PLPs were formed in one step, where all lipid constituents were incorporated at the time of the lipid nanoparticle assembly.

In another embodiment, the CTP-PLPs were formed via post-insertion. Unlike the one-step pre-insertion, post-insertion required functional ligands to be incorporated into pre-formed base lipid nanoparticles. Base PLPs were assembled first without the incorporation of collagen-targeting peptides, and initially purified from EtOH via 2 h dialysis against PBS at 4° C. DSPE-PEG-CTPs, as described in Example 1, were dissolved in CHCl₃ and dried under inert gas into a separate lipid film. Pre-formed base PLPs were then incubated with the lipid film at 37° C. under constant vortexing for 2 h to allow micellar transfer. The formed CTP-PLPs were further purified via 24 h dialysis against PBS at 4° C. and extruded.

### Example 4: Cell-Penetrating Peptide-Conjugated Lipid Nanoparticle (R8-PLP) Assembly

Cell-penetrating peptide-conjugated lipid nanoparticles were assembled and used as control groups. R8 (SEQ ID NO. 7 or SEQ ID NO. 8) was covalently attached to stearic acid to form the cell-penetrating lipid STR-R8. An aqueous solution of STR-R8 was reconstituted. A reaction vessel, for example a 2.5 mL dram vial, was washed with DEPC-treated Millipore water and dried under inert gas. 10 mol % of STR-R8 was charged to the reaction vessel and the solution dried under a stream of inert gas to form a dry STR-R8 film. Lipids 1,2-dioleoyl-sn-glycero-3-phosphocholine (DOPC) and ovine cholesterol (Chol) were mixed at a mole ratio of 7:3. Optionally, about 0.1 mol %-about 0.5 mol % Rho-PE or about 0.1 mol % -about 0.5 mol % Cy7-PE was added into the mixture for fluorescent labeling. The mixture was added into the reaction vessel containing the STR-R8 film and the solvent removed under a stream of inert gas to form a dry lipid film. The reaction vial was placed under vacuum to remove all traces of organic solvent. At this point the reaction vessel could be stored at 4° C. for further use. To the reaction vessel was added absolute ethanol at a volume that constitutes 40% of the final reaction volume. The vessel was vortexed for 10 seconds then transferred to an incubator held 40° C. while the vessel was shaken at 200 RM for 1 hour. The reaction vessel was vortexed and centrifuged to spin down the ethanol solution containing the dissolved lipids. See, International Publication No. WO 2020/023311, which is incorporated herein by reference.

### Example 5: Multi-Functional Lipid Nanoparticle (CTP-R8-PLP) Assembly

In this Example, dual ligand multi-functional lipid nanoparticles were formed. The dual ligands conjugated to the lipid nanoparticles comprised a cell-penetrating peptide and a collagen-targeting peptide. The cell-penetrating peptide R8 (SEQ ID NO. 7 or SEQ ID NO. 8) was covalently attached to stearic acid to form the cell-penetrating lipid STR-R8. The collagen-targeting peptide-conjugated lipid DSPE-PEG-CTP was formed as described in Example 1, where the CTP and DSPE-PEG-DBCO were conjugated via click chemistry.

An aqueous solution of STR-R8 was reconstituted. A reaction vessel, for example a 2.5 mL dram vial, was washed with DEPC-treated Millipore water and dried under inert gas. 10 mol % of STR-R8 was charged to the reaction vessel and the solution dried under a stream of inert gas to form a dry STR-R8 film. Lipids 1,2-dioleoyl-sn-glycero-3-phosphocholine (DOPC) and Ovine cholesterol (Chol) were mixed at a mole ratio of 7:3. 5 mol % of DSPE-PEG-CTP was added into the mixture of DOPC and Chol. Optionally, about 0.1 mol %-about 0.5 mol % Rho-PE or about 0.1 mol %-about 0.5 mol % Cy7-PE was added into the mixture for fluorescent labeling. The mixture of DOPC, Chol and DSPE-PEG-CTP was added into the reaction vessel containing the dry R8 film and the solvent removed under a stream of inert gas to form a dry lipid film. The reaction vial was placed under vacuum to remove all traces of organic solvent. and dried under inert gas and vacuum to remove the remaining solvent. The dry lipid film was then resuspended in 100% molecular grade EtOH and injected dropwise into 10 mM Tris-HCl at pH 8.0 and a 2:3 EtOH:aqueous volume ratio, under constant vortexing at room temperature. Lipid nanoparticles were purified from EtOH via 24 h dialysis against PBS at 4° C. and extruded as described above. As such, the dual ligand lipid nanoparticles were formed in one step, where all lipid constituents were incorporated at the time of the lipid nanoparticle assembly.

### Example 6: Encapsulation of Biologically Active Ingredients in Multi-Functional Lipid Nanoparticles (CTP-R8-PLP)

In this Example, multi-functional lipid nanoparticles as described in Example 5 were encapsulated with biologically active ingredients. siRNA for GAPDH gene silencing was used as an example biologically active ingredient. 50 μg of siRNA was diluted with 600 μL of 10 mM Tris-HCl buffer containing 5-50 mM CaCl₂ at pH 8.0. The siRNA solution was added such that the volume of liquid constitutes 60% of the final reaction volume. The ratio of lipid to siRNA could range from about 20:1 to about 100:1 weight/weight, dependent on concentration requirements of downstream applications. The multi-functional lipid nanoparticles (CTP-R8-PLPs) as described in Example 5 were added dropwise to the aqueous solution of siRNA with effective mixing at a rate selected from the range of about 0.1 mL/minute to about 0.8 mL/minute. The resulted mixture was dialyzed in at least 500 excess volume of phosphate buffered solution (PBS) at pH 7.4 for 18 hours at 4° C. with stirring to remove any free siRNA and organic solvent. The PBS was changed at 2-4 hours and at 6-8 hours during the dialysis. The resulting ligand nanoparticles encapsulated with siRNA were stored at approximately 4° C. in nuclease-free tubes or vessels. Optionally, these nanoparticles could be extruded through a polycarbonate membrane.

### Example 7: Characterization of Lipid Nanoparticles

The size and zeta potential of the lipid nanoparticles described in Examples 2-6 were measured in triplicate by dynamic light scattering and electrophoretic mobility on the Zetasizer Nano ZS instrument. The morphology and lamellarity were investigated by scanning transmission electron microscope (STEM) using a negative-stain method. In particular, the lipid nanoparticles were applied dropwise to a carbon film coated copper grid and allowed to air dry. The formed lipid nanoparticle film was subsequently stained with 2% phosphotungstic acid and air-dried for 1 minute at room temperature. The stained film was then visualized with Zeiss Auriga 40 STEM scope, and images were acquired by SmartSEM image acquisition software.

The multi-functional lipid nanoparticles have physical characteristics desirable for clinical translation. Dynamic light scattering results confirmed a narrow size distribution at <50 nm following EtOH injection and extrusion. Zeta-potentials demonstrated a low positive charge. Table 2 provides the characteristics of multi-functional lipid nanoparticles (CTP-R8-PLPs) and comparison with control groups including base lipid nanoparticles (PLPs) and collagen-targeting peptide-conjugated lipid nanoparticles (CTP-PLPs). The formed CTP-R8-PLP particles had a substantially uniform size of 47.5 nm with a narrow size distribution, similar to PLPs and CTP-PLPs. The polydispersity index (PDI) of less than 0.2 and the semi-neutral charge also demonstrated the stability of CTP-R8-PLP particles. These physical characteristics make CTP-R8-PLP particles conducive to positive biocompatibility profiles, evasion of RES systemic clearance, and improved half-life and stability. These characteristics also indicate the CTP-R8-PLP particles can function as an efficient drug delivery vehicle, suggesting a high potential for clinical translation of the technology described herein.

TABLE 2
Lipid Nanoparticle Characteristics

| Liposome | Size | | Charge |
|---|---|---|---|
| Class | (nm) | PDI | (Zeta-potential) |
| PLP | 46.6 ± 1.7 | 0.13 ± 0.01 | 10.9 ± 1.0 |
| CTP-PLP | 45.3 ± 2.3 | 0.19 ± 0.03 | 11.9 ± 0.5 |
| CTP-R8-PLP | 47.5 ± 2.1 | 0.17 ± 0.02 | 16.0 ± 2.1 |

### Example 8: Lipid Nanoparticle Cell Association

### Vascular Smooth Muscle Cell Culture

Human aortic smooth muscle cells (HASMC) were obtained from LifeLine Cell Technology as cryopreserved primary cultures. Cells were plated at 1.5×10⁵ cells/well (6-well plate) to assay delivery efficiency via gene expression analyses or at 4×10⁴ cells/well (24-well plate) for cell association experiments. Cells were incubated at 37° C. in an environment of 5% CO₂ and 95% humidity and grown to 60-80% confluency in VascuLife growth medium (VascuLife Basal Medium+VascuLife smooth muscle cell supplement kit+gentamyocin/amphotericin). Prior to experimental use, cells were made quiescent overnight in Dulbecco's Modified Eagle Medium containing gentamyocin/amphotericin.

### Lipid Nanoparticle Cell Association

Cell association assays were performed to demonstrate both lipid nanoparticle tracking and lipid nanoparticle-mediated cellular delivery. The lipid nanoparticles described in Examples 2-5 with the addition of 0.1 mol % Rho-DOPE were used for cell association assays. At ~80% confluency, HASMCs were treated with base lipid nanoparticles (PLPs), collagen-targeting peptide-conjugated nanoparticles (CTP-PLPs) and multi-functional lipid nanoparticles conjugated with collagen-targeting peptide and cell-penetrating peptide (CTP-R8-PLPs), respectively, at 0.2 mM total lipid in DMEM. After 24 hours of treatment, the cells were washed twice in PBS buffer, lysed with 1% Triton X-100, and centrifuged at 12,000 RPM for 5 minutes at 4° C. to remove cell debris. Cell lysates (100 μL) were plated in duplicate in 96-well plates, and cell association was determined by fluorometry of Rhodamine at 575 nm. Cell association was determined by mean arbitrary fluorescence units (AFU) of each sample, minus baseline fluorescence of non-treated controls receiving no Rhodamine source within each experimental replicate. For qualitative analysis fluorescent microscopy images were acquired using a Texas Red fluorescent filter at 400× under 400 millisecond exposure across all groups.

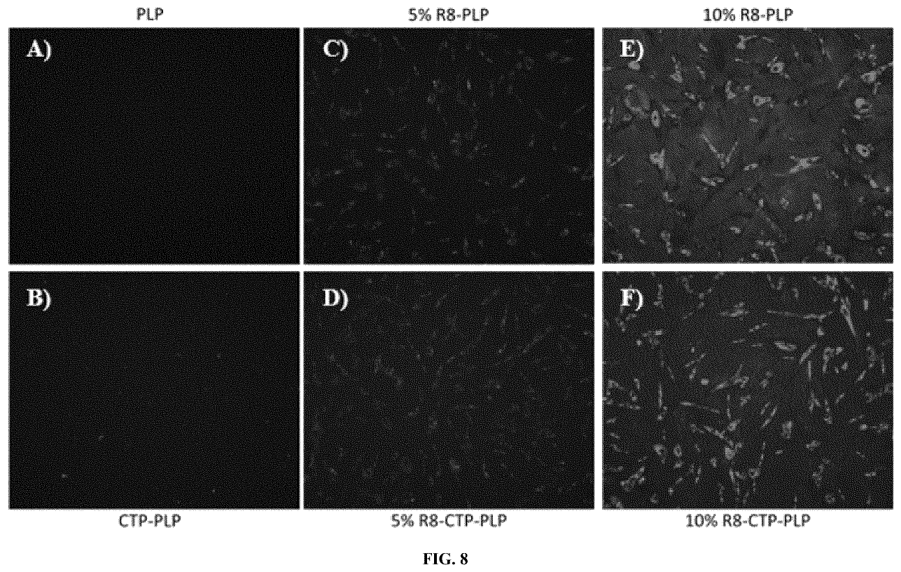

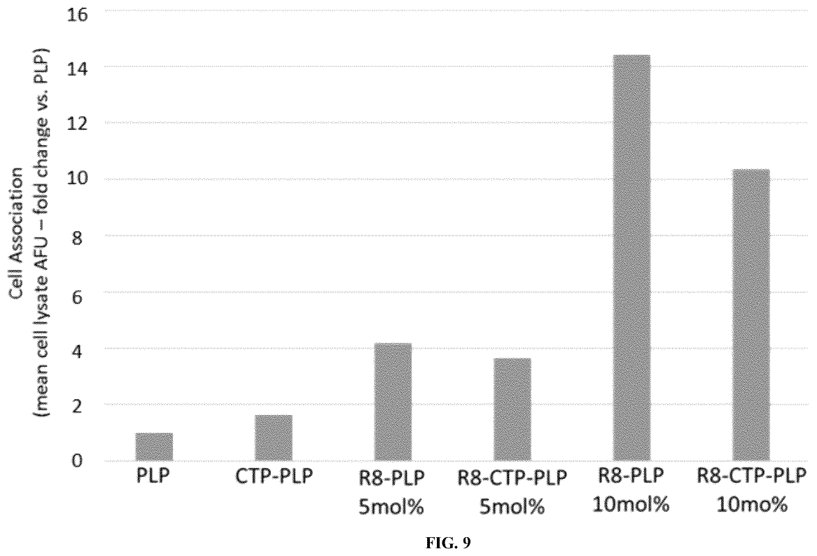

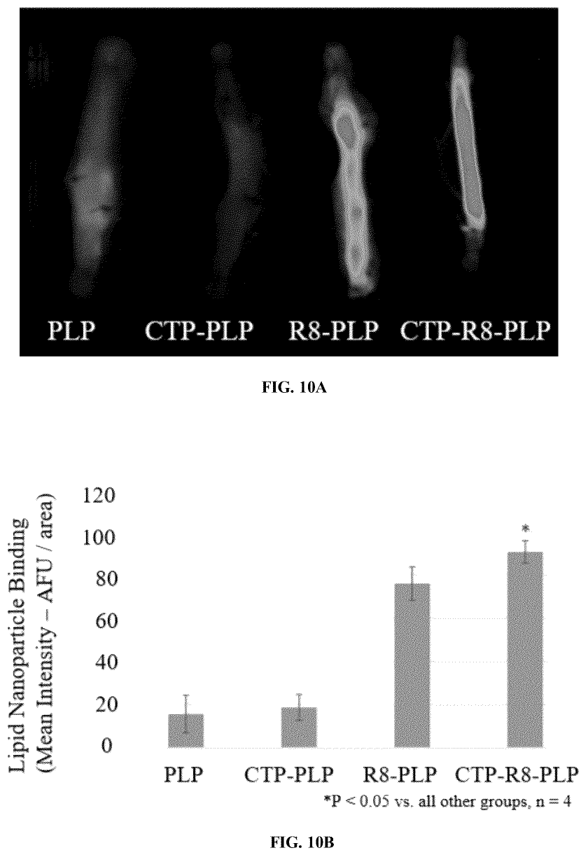

The multi-functional lipid nanoparticles conjugated with cell-penetrating peptides and collagen-targeting peptides (e.g., CTP-R8-PLPs) showed significantly improved targeted binding to human aortic smooth muscle cells. FIG. 8 illustrates the fluorescent images of HASMC after treated with base lipid nanoparticles PLPs (FIG. 8A), collagen-targeting peptide-conjugated lipid nanoparticles CTP-PLPs (FIG. 8B), 5 mol % cell-penetrating peptide-conjugated lipid nanoparticles R8-PLPs (FIG. 8C), 5 mol % multi-functional lipid nanoparticles conjugated with collagen-targeting peptide and cell-penetrating peptide CTP-R8-PLPs (FIG. 8D), 10 mol % R8-PLPs (FIG. 8E) and 10 mol % CTP-R8-PLPs (FIG. 8F), respectively. The fluorescent images qualitatively validated that multi-functional lipid nanoparticles substantially increased cellular association. For example, CTP-R8-PLPs increased the HASMC cellular association by 186±38-fold over PLP controls, and by 37±14-fold in CTP-PLP controls. FIG. 9 illustrates the quantification of HASMC cellular association using different types of lipid nanoparticles. The multi-functional nanoparticles CTP-R8-PLPs showed a high affinity for association with vascular cell types in vitro. The substantial increase in cellular association and improved ability of fluorescent labeling, allow these multi-functional nanoparticles to be optimized for imaging and drug delivery in vascular tissues.

### Example 9: Lipid Nanoparticle Binding to Vessel Explants Under Ex Vivo Flow

The multi-functional lipid nanoparticles showed increased binding affinity for human vessel explants under ex vivo flow. Human vessel explants were obtained from amputated limbs from the operating room. Vessels were sterilely extracted by fine micro-dissection, flushed with sterile saline, cut transversely into ~6 cm segments, canulated to perfusion bioreactor flow chambers, and maintained ex vivo in bioreactor culture with pulsatile lumen perfusion with PBS for short term viability maintenance. LNP formulations were assembled as described above. Independent vessel explants were perfused through the bioreactor chamber with LNP groups for 30 minutes with 50 μM total lipid for vessel wall binding. Vessels were then visualized under near-infrared imaging to detect lipid nanoparticles that bind to the pathological vessel walls. The lipid nanoparticle binding was quantified as mean intensity of bound Cy7-labeled lipid per vessel area, normalized to background of non-perfused harvested vessel segments.

FIG. 10A illustrates fluorescent images of human vessel explants after perfused with base lipid nanoparticles PLPs, collagen-targeting peptide-conjugated lipid nanoparticles CTP-PLPs, cell-penetrating peptide-conjugated lipid nanoparticles R8-PLPs and multi-functional lipid nanoparticles conjugated with collagen-targeting peptide and cell-penetrating peptide CTP-R8-PLPs. FIG. 10B illustrates the quantification of lipid nanoparticles binding to the vessel explants. Both the fluorescent images and quantification demonstrate CTP-R8-PLPs showed an increased binding affinity for human lower extremity vessels under simulated vascular flow, compared to control groups.

### Example 10: Lipid Nanoparticle Binding to Vascular Tissues In Vivo

The multi-functional lipid nanoparticles showed increased binding affinity for vascular tissues in vivo using an animal model. Sprague-Dawley rats underwent localized vascular infusion of different types of lipid nanoparticles and PBS control in a surgically isolated area of the left common carotid artery. Briefly, rats were anesthetized with vaporized 1-5% isoflurane and the left common carotid artery exposed via midline neck incision. Once proximal control of the common carotid and distal control of the internal and external branches were secure, access to the lumen was obtained through a small nick in the external carotid. The internal catheter of a 4F introducer was passed through to the common carotid artery, and the isolated section cleared with a sterile saline wash and perfused with LNPs for 5 min. Residual perfusion solution was flushed with sterile saline, the external carotid ligated, blood flow through the carotid system restored. Rats were euthanized after 5 min of systemic flow and their carotid arteries procured for imaging. An additional cohort or rats will undergo LBP in the same manner and be euthanized at 24 h, 48 h, or 72 h post-LBP to assay vessel viability and efficiency of transfection. A subset of rats underwent vascular injury of the common carotid via balloon angioplasty (BA) immediately prior to lipid nanoparticle infusion. The standard BA procedure was performed where rats were anesthetized and a 2F balloon catheter passed through a small nick in the external carotid artery to the common carotid artery, the balloon distended to 2 atm of pressure with saline, and passed anti- and retro-grade through the common carotid to denude the endothelium. This was repeated three times, after which the catheter was removed and the LNP perfusion described above was carried out. all cases, the rats were continuously infused with lipid nanoparticles at 50 μM total lipid using a hollow 2F catheter for 5 minutes. Complete systemic flow was subsequently restored and allowed to persist for 5 minutes prior to euthanasia. Carotid vessels were harvested and fluorescently imaged at near-infrared for 30 seconds. The lipid nanoparticle binding was quantified as mean intensity of bound Cy7-labeled lipid per vessel area, normalized to background of non-perfused right carotid arteries.

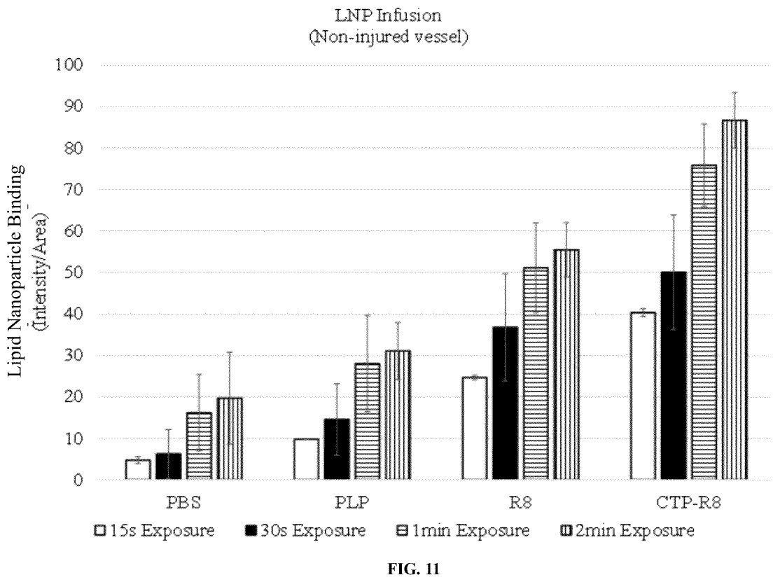

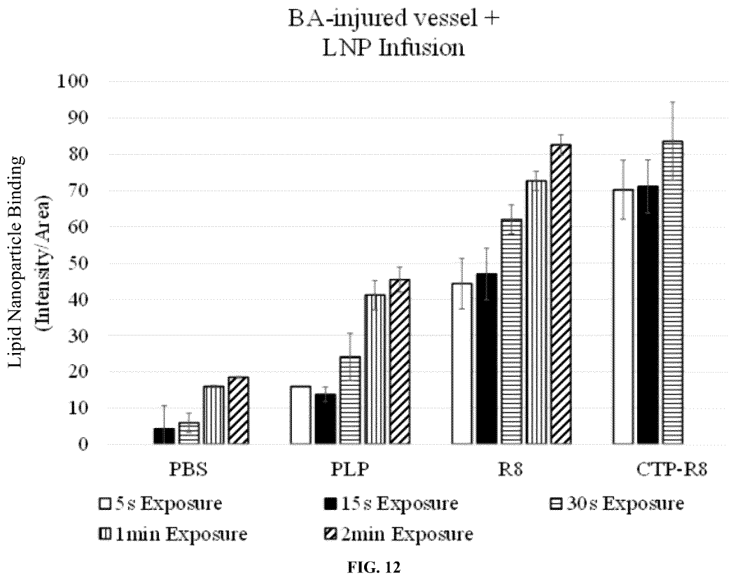

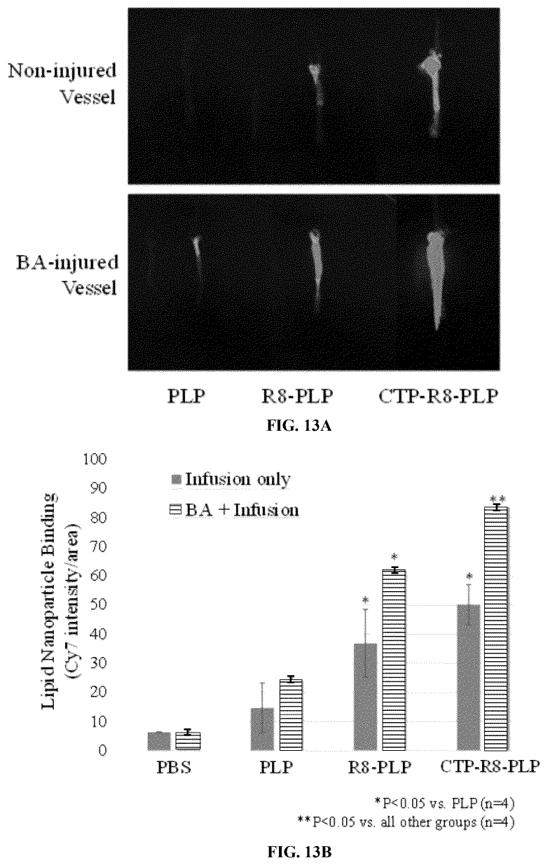

FIG. 11 illustrates the lipid nanoparticle binding to non-injured vessels via localized vascular infusion using PBS control, base lipid nanoparticles PLPs, cell-penetrating peptide-conjugated lipid nanoparticles R8-PLPs and multi-functional lipid nanoparticles conjugated with collagen-targeting peptide and cell-penetrating peptide CTP-R8-PLPs. CTP-R8-PLPs bind to non-injured vessels. Binding affinity is strong enough to be detected after as short as 15 second exposure under near-infrared imaging analysis. The binding efficiency and efficacy of the CTP-R8-PLP particles exceed all control groups. FIG. 12 illustrates the lipid nanoparticle binding to BA-injured vessels via localized vascular infusion using base lipid nanoparticles PLPs, collagen-targeting peptide-conjugated lipid nanoparticles CTP-PLPs, cell-penetrating peptide-conjugated lipid nanoparticles R8-PLPs and multi-functional lipid nanoparticles conjugated with collagen-targeting peptide and cell-penetrating peptide CTP-R8-PLPs. The CTP-R8-PLP particles bound to BA-injured vessels with an affinity strong enough for detection after as short as 5 second exposure. FIG. 13A illustrates fluorescent images of non-injured and BA-injured vessels via localized vascular infusion using base lipid nanoparticles PLPs, cell-penetrating peptide-conjugated lipid nanoparticles R8-PLPs and multi-functional lipid nanoparticles conjugated with collagen-targeting peptide and cell-penetrating peptide CTP-R8-PLPs. FIG. 13B illustrates the quantification of the lipid nanoparticle binding to non-injured and BA-injured vessels normalized at a 30 second exposure. Both the fluorescent images and quantification demonstrated CTP-R8-PLPs are significantly more efficacious in binding injured and non-injured vessels compared to control groups.

### Example 11: Lipid Nanoparticle Encapsulate Delivery and Response Via Gene Expression Analysis

The multi-functional lipid nanoparticles were encapsulated with siRNA, as described in Example 6. The siRNA encapsulation was determined using Quant-iT RiboGreen RNA Assay Kit (Thermo Fisher Scientific). The lipid nanoparticles were solubilized in 1% Triton X-100 at 37° C. for 15 minutes to release encapsulated siRNA, mixed 1:1 with RiboGreen reagent for fluorescent labeling of siRNA. The fluorescent emission was measured at 525 nm on a GloMax Multi Instrument. Fluorescence units of solubilized lipid nanoparticles were then compared to a known standard curve of siRNA in 1% Triton X-100 to determine the amount of siRNA being encapsulated in the lipid nanoparticles. The encapsulation efficiency (EE %) of siRNA for each type of lipid nanoparticles was calculated as (μg siRNA encapsulate/50 μg total siRNA)×100.

To demonstrate lipid nanoparticle-mediated delivery and delivery-mediated cellular response, the lipid nanoparticles as described in Examples 2-6 were assembled and encapsulated with GAPDH siRNA. GAPDH siRNA was used to knock down human GAPDH mRNA expression. HASMCs were transfected with base lipid nanoparticles (PLPs), collagen-targeting peptide-conjugated nanoparticles (CTP-PLPs) and multi-functional lipid nanoparticles conjugated with collagen-targeting peptide and cell-penetrating peptide (CTP-R8-PLPs), respectively, with 400 nM siRNA in serum-free DMEM. After 24 hours of transfection, DMEM was removed, and cells collected to assay lipid nanoparticle-mediated GAPDH silencing via quantitative Polymerase Chain Reaction (qPCR). Total RNA was isolated using Ambion Paris Kit according to manufacturer's instructions, and 200 ng was converted to cDNA using the High Capacity RNA-to-cDNA Master Mix. 2 μL of cDNA transcripts were then amplified by qPCR using the TaqMan Gene Expression Master Mix and predesigned TaqMan Gene Expression Assays specific for human GAPDH on the StepOne PCR instrument. The comparative cycle threshold method was used to determine relative quantity of GAPDH mRNA in lipid nanoparticle treated samples compared with non-treated controls. All mRNA amounts were normalized to 18S ribosomal RNA as an endogenous control.

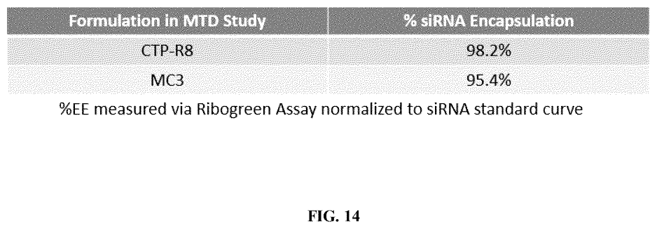

FIG. 14 illustrates siRNA encapsulation efficiency of lipid nanoparticles PLP, collagen-targeting peptide-conjugated lipid nanoparticles CTP-PLP and multi-functional lipid nanoparticles conjugated with collagen-targeting peptide and cell-penetrating peptide CTP-R8-PLP. The CTP-R8-PLP particles provided over 5-fold increase in the siRNA encapsulation efficiency.

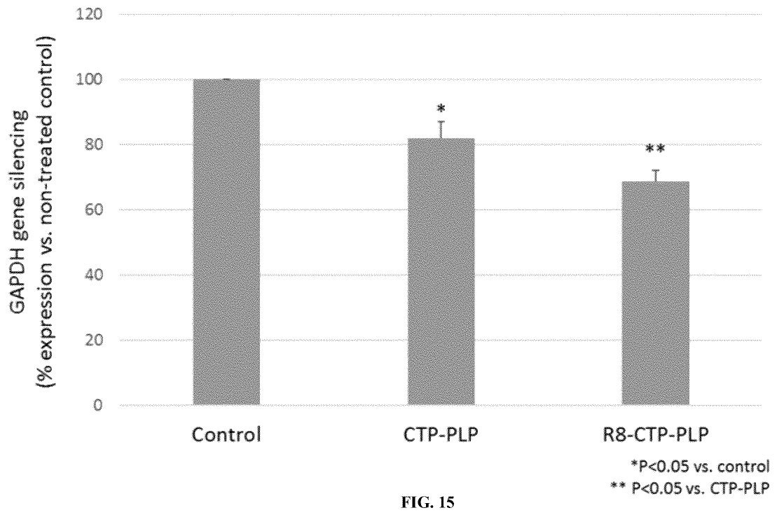

FIG. 15 compares the GAPDH gene silencing effect using collagen-targeting peptide-conjugated lipid nanoparticles CTP-PLP and multi-functional lipid nanoparticles conjugated with collagen-targeting peptide and cell-penetrating peptide CTP-R8-PLP. The CTP-R8-PLP particles significantly increased the siRNA delivery and thus, enhanced gene silencing compared to control groups.

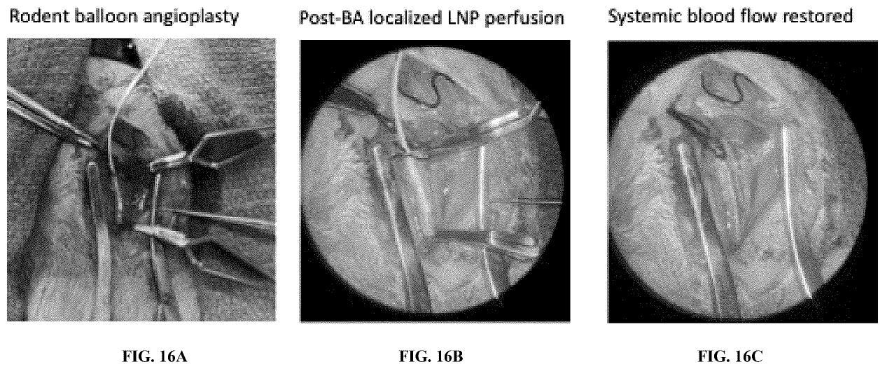

FIGS. 16A-16C depict in vivo balloon angioplasty and localized double balloon perfusion technique for lipid nanoparticle LNP perfusion. FIG. 16A illustrates a rodent that underwent balloon angioplasty prior to lipid nanoparticle perfusion. FIG. 16B illustrates localized lipid nanoparticle LNP perfusion post balloon angioplasty. FIG. 16C illustrates the systemic blood flow is restored after lipid nanoparticle LNP perfusion.

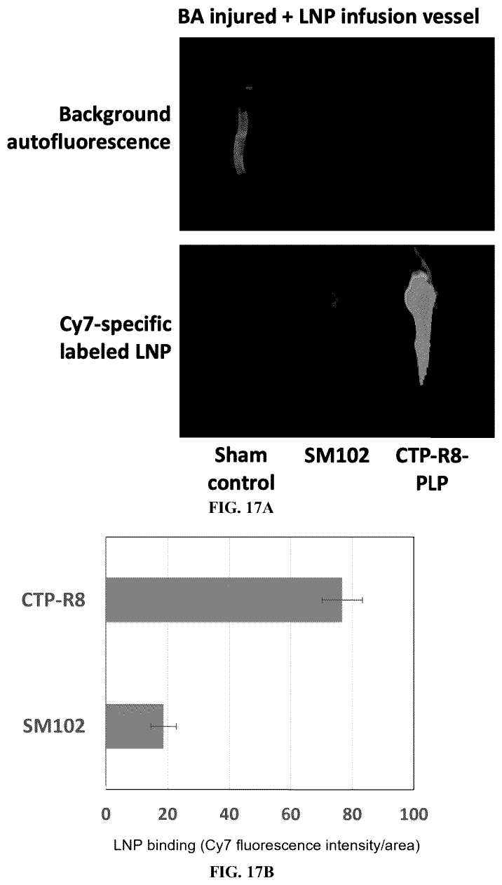

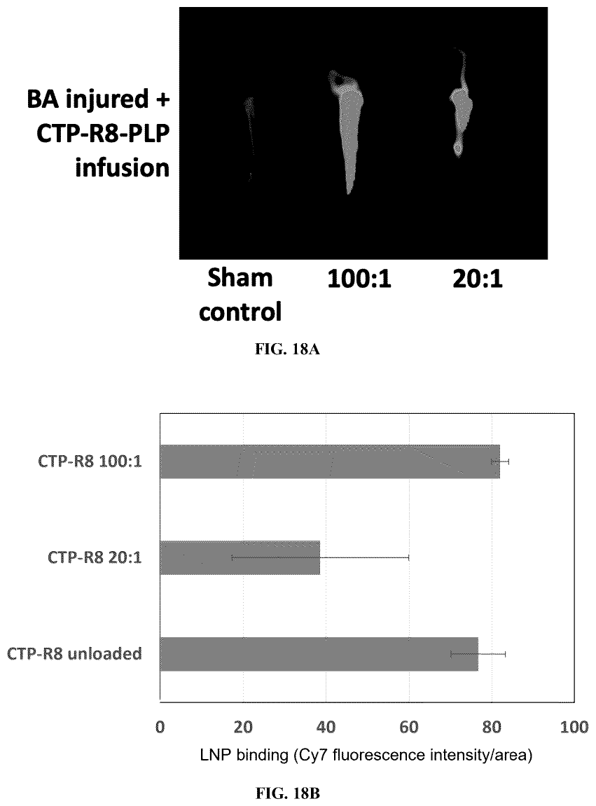

FIGS. 17A and 17B illustrate vessel wall binding of multi-functional lipid nanoparticles conjugated with collagen-targeting peptide and cell-penetrating peptide CTP-R8-PLP compared to SM102, a clinically relevant pharmaceutical formulation. FIG. 17A illustrates fluorescent images of BA-injured vessels via localized vascular infusion using Sham control, CTP-R8-PLP, and SM102. FIG. 17B illustrates the lipid nanoparticle binding to BA-injured vessels via localized vascular infusion using multi-functional lipid nanoparticles conjugated with collagen-targeting peptide and cell-penetrating peptide CTP-R8-PLP and SM102. The heatmap as illustrated in FIG. 17A shows Cy7-labeled lipid nanoparticles LNPs binds to vessels. Non injured vessels with no disease/pathology serve as the sham control to normalize for endogenous background vessel fluorescence. All other groups were subject to common carotid via balloon angioplasty (BA) immediately prior to LNP infusion. Vessels were subjected to 5 min LNP perfusion with CTP-R8-PLPs and to a standardized and clinically relevant pharmaceutical formulation SM102, then systemic flow restored for 5 min prior to euthanasia and vessel imaging. CTP-R8-PLP were significantly more efficacious in binding injured vessels compared to the clinically relevant pharmaceutical formulation SM102.

FIGS. 18A and 18B depict the optimization of lipid with siRNA weight to weight loading parameters for enhanced CTP-R8-PLP binding based on carrying capacity data of other empirically derived LNP formulations. FIG. 18A illustrates fluorescent images of BA-injured vessels via localized vascular infusion using Sham control, CTP-R8-PLP with a ratio of lipid to siRNA of 100:1, and a ratio of 20:1. FIG. 18B illustrates the lipid nanoparticle binding to BA-injured vessels via localized vascular infusion using unloaded CTP-R8-PLP, CTP-R8-PLP with a ratio of lipid to siRNA of 100:1, and with a ratio of 20:1. The heatmap as illustrated in FIG. 18A shows Cy7-labeled LNPs bind to vessels. Non injured vessels with no disease/pathology serve as the sham control to normalize for endogenous background vessel fluorescence. All other groups were subject to common carotid via balloon angioplasty (BA) immediately prior to LNP infusion. Vessels were subjected to LNP perfusion with CTP-R8-PLPs, both unloaded and loaded with siRNA cargo at 20:1 and 100:1 lipid:siRNA weight to weight assembly parameters. Based on other LNP formulations being empirically derived, it was found that lipid:siRNA loading parameters impact internal carrying capacity vs. outer membrane association of siRNA cargo. Because 20:1 assembly parameters results in ~50/50 internalization vs. association, while 100:1 results in almost total internalization of siRNA cargo, it was established that LNP formulations with internalization of siRNA cargo would have enhanced binding affinity, whereas surface bound LNP peptides have a greater capacity for their mechanism of action. As illustrated in FIGS. 17A and 17B, CTP-R8-PLP loaded at 100:1 lipid:siRNA were significantly more efficacious in binding injured vessels compared to 20:1. Therefore, CTP-R8-PLP loaded at 100:1 lipid:siRNA was demonstrated as an efficient targeted drug carrier.

### Example 12: Toxicity Study of Lipid Nanoparticle Formulations

In this example, toxicity of lipid nanoparticle formulations made with ionic lipids MC-3 were evaluated against CTP-R8-PLP in a maximum tolerable dose (MTD) study.

Experimental Conditions: The lipid particles were dosed via IV route once in C57BL6/J female mice. Blood was collected in life and terminal for alanine aminotransferase (ALT)/aspartate aminotransferase (AST) analysis. All study procedures were conducted by qualified personnel at Crown Bioscience San Diego (16550 West Bernardo Dr. #525, San Diego, CA 92127 USA), and were in accordance with Crown Bioscience San Diego Standard Operating Procedures. Table 3 lists information of treatment groups used in this study. Table 4 lists test and control articles (e.g., CTP-R8-PLP, MC3 lipid LNP) used in the study. The articles were shipped frozen, thawed at room temperature, and mixed gently by pipette or by inverting the vial for 10 times. Dilution of the LNP stock for lower dose was done using PBS buffer.

TABLE 3
Information of treatment groups used in toxicity study

| Species | Mice |
|---|---|
| Strain | C57BL6 |
| Source | Jackson |
| Age/DOB | DOB 7 Nov. 2023 |
| | 9 week old |
| Sex | Female |
| Number on Study | 24 |
| Housing per Cage | 4 |

TABLE 4
Test and control articles used in toxicity study

| Name | Vehicle |
|---|---|
| Orion CTP-R8 (CTP-R8-PLP) | PBS pH 7.4 + 8.7% sucrose |
| MC3 lipid LNP | PBS pH 7.4 + 8.7% sucrose |

Part of the test and control articles were evaluated to see whether the freezing and thawing processes had impact on their corresponding characteristics. Samples were formulated on Day 0 and dialyzed and sterile filtered on Day 1 before analytical methods were performed. During downstream processing on Day 1, samples were diluted with sucrose to 8.7% sucrose+PBS. Samples were frozen in liquid nitrogen and stored at −20 C.°, then thawed on Day 13 and measured for size and polydispersity index (PDI) for physical stability. The samples were then stored at C° and tested again for size and PDI 3 weeks later. Table 5 lists average size of test and control articles. Table 6 lists polydispersity index (PDI) of test and control articles.

TABLE 5
Average size (Z-ave, d · nm) of test and control articles

| | Day 1 - Before | Day 13 - After | Day 35 - After thawing and |
|---|---|---|---|
| Sample | Freezing | Thawing | storage at 4 C. ° for 3 weeks |
| R8-CTP | 51.3 | 89.2 | 87.3 |
| MC3 | 107 | 154.1 | 167 |

TABLE 6
Polydispersity index (PDI) of test and control articles

| | Day 1 - Before | Day 13 - After | Day 35 - After thawing and |
|---|---|---|---|
| Sample | Freezing | Thawing | storage at 4 C. ° for 3 weeks |
| R8-CTP | 0.092 | 0.127 | 0.1388 |
| MC3 | 0.01 | 0.0257 | 0.02241 |

Table 7 lists treatment groups in toxicity study with 24 mice enrolled. All animals were randomly allocated to 6 different study groups. Randomization was performed in the Study Log software on day −1 (i.e., 9 Jan. 2024) based on body weight. Average body weight for each group was 19.62±0.65 grams at randomization. Table 8 lists reference days of the toxicity study. For clinical chemistry experiments, 63 serum samples were collected from in-life and terminal bleeds and used for clinical chemistry. As listed in Table 9, 70 μL serum (diluted serum for in life bleeds) were used to determine ALT and AST. Sample analysis was performed by Beckman Coulter AU480 Clinical Chemistry Analyzer. Raw data will be exported and summarized in Microsoft Excel with optional group statistics. Table 10 lists analytes and corresponding dynamic ranges.

TABLE 7
Treatment groups in toxicity study.

| | | | | Dosing | | Dose |
|---|---|---|---|---|---|---|
| | | | Dose | Frequency & | Dose Level | Volume |
| Group | N | Treatment | Route | Duration | (mg/kg) | (mL/kg) |
| 1 | 4 | CTP-R8-PLP - High | Tail/IV | Two injections | 2 (combined 4 | 10 |
| | | Dose (2 mg/kg) | | | for 2 injections | |
| 2 | 4 | CTP-R8-PLP - | Tail/IV | Single injection | 1 | 5 |
| | | Medium Dose (1 | | | | |
| | | mg/kg) | | | | |
| 3 | 4 | CTP-R8-PLP - Low | Tail/IV | Single injection | 0.25 | 5 |
| | | Dose (0.25 mg/kg) | | | | |
| 4 | 4 | MC3 lipid - High | Tail/IV | Two injections | 2 (combined 4 | 10 |
| | | Dose (2 mg/kg) | | | for 2 injections | |
| 5 | 4 | MC3 lipid - | Tail/IV | Single injection | 1 | 5 |
| | | Medium Dose (1 | | | | |
| | | mg/kg) | | | | |
| 6 | 4 | MC3 lipid - | Tail/IV | Single injection | 0.25 | 5 |
| | | Low Dose (0.25 | | | | |
| | | mg/kg) | | | | |

TABLE 8
Reference days of the toxicity study

| | Study Day | Date |
|---|---|---|
| Study Start | 0 | 10 Jan. 2024 |
| Dosing Start | 0 | 10 Jan. 2024 |
| Dose End | 0 | 10 Jan. 2024 |
| End of Study | 7 | 17 Jan. 2024 |

TABLE 9
Clinical chemistry parameters in toxicity study

| Brand | Model | Sample | N | Analytes | Processing |
|---|---|---|---|---|---|
| Beckman | AU480 | Serum | 63 | ALT, AST | Terminal, +In-Life |
| Coulter | Chemistry | | | | Bleeds at D −1, 24 hr |
| | Analyzer | | | | & 48 hr post-dosing |

TABLE 10
List of analytes and corresponding dynamic ranges

| Analyte | Dynamic Range | units |
|---|---|---|
| AST | 3 to 1000 | U/L |
| ALT | 3 to 500 | U/L |

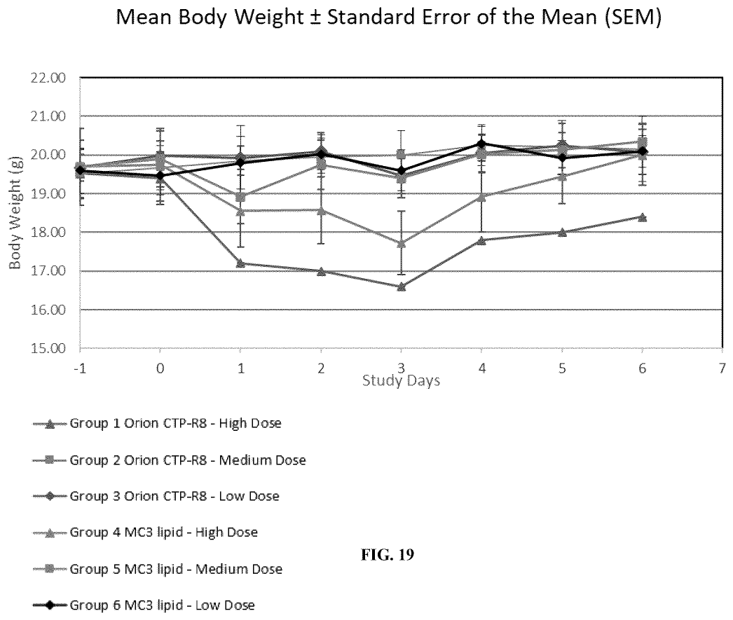

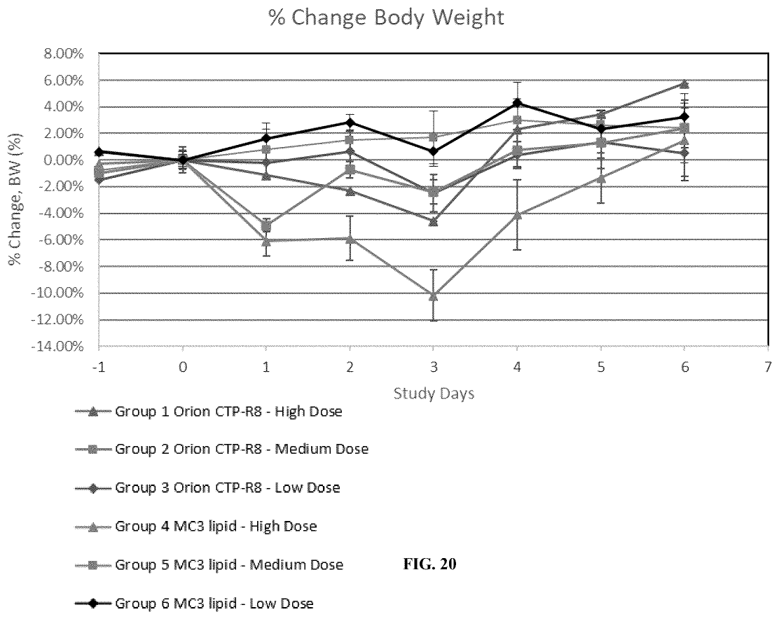

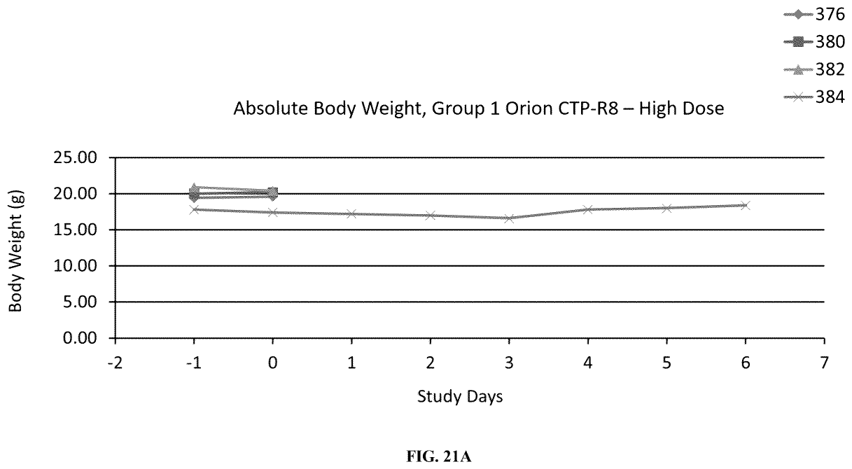

Body weight measurements: FIG. 19 shows body weight measurements of treatment groups across study days. Table 11 lists mean body weight (g) of each group over study days. Table 12 lists standard error mean of each group over study days. FIG. 20 shows body weight change of treatment groups across study days, where body weight of each group on Day 0 was used as initial value. Table 13 lists body weight change of each group over study days using body weight on Day 0 and Day 1 as initial value. It was demonstrated the body weight of each treatment group remained stable during the study. The use of high dose of both CTP-R8-PLP (group 1) and MC3 (group 4) caused a decrease in body weight. The treatment groups that received CTP-R8-PLP (i.e., groups 1-3) had a smaller body weight change compared to the groups that received MC3 lipid.

TABLE 11
Mean body weight (g) of each group over study days.

| Group | Day −1 | Day 0 | Day 1 | Day 2 | Day 3 | Day 4 | Day 5 | Day 6 |
|---|---|---|---|---|---|---|---|---|
| CTP-R8-PLP - High Dose | 19.53 | 19.40 | 17.20 | 17.00 | 16.60 | 17.80 | 18.00 | 18.40 |
| CTP-R8-PLP - Medium | 19.53 | 19.68 | 19.85 | 19.95 | 20.00 | 20.25 | 20.20 | 20.15 |
| Dose | | | | | | | | |
| CTP-R8-PLP - Low Dose | 19.68 | 19.98 | 19.93 | 20.10 | 19.48 | 20.05 | 20.25 | 20.08 |
| Group 4 MC3 lipid - High | 19.70 | 19.75 | 18.55 | 18.58 | 17.73 | 18.93 | 19.45 | 20.00 |
| Dose | | | | | | | | |
| Group 5 MC3 lipid - | 19.70 | 19.90 | 18.93 | 19.75 | 19.40 | 20.03 | 20.13 | 20.35 |
| Medium Dose | | | | | | | | |
| Group 6 MC3 lipid - Low | 19.60 | 19.48 | 19.80 | 20.03 | 19.60 | 20.30 | 19.93 | 20.10 |
| Dose | | | | | | | | |

TABLE 12
Standard error mean of each group over study days.

| Group | Day −1 | Day 0 | Day 1 | Day 2 | Day 3 | Day 4 | Day 5 | Day 6 |
|---|---|---|---|---|---|---|---|---|
| Group 1 CTP-R8-PLP - | 0.65 | 0.69 | 0.00 | 0.00 | 0.00 | 0.00 | 0.00 | 0.00 |
| High Dose | | | | | | | | |
| Group 2 CTP-R8-PLP - | 0.62 | 0.57 | 0.90 | 0.50 | 0.64 | 0.52 | 0.70 | 0.84 |
| Medium Dose | | | | | | | | |
| Group 3 CTP-R8-PLP - | 0.35 | 0.38 | 0.30 | 0.48 | 0.48 | 0.49 | 0.57 | 0.59 |
| Low Dose | | | | | | | | |
| Group 4 MC3 lipid - | 0.99 | 0.93 | 0.92 | 0.87 | 0.83 | 0.92 | 0.71 | 0.78 |
| High Dose | | | | | | | | |
| Group 5 MC3 lipid - | 0.69 | 0.72 | 0.70 | 0.64 | 0.51 | 0.49 | 0.46 | 0.46 |
| Medium Dose | | | | | | | | |
| Group 6 MC3 lipid - | 0.58 | 0.50 | 0.69 | 0.49 | 0.53 | 0.44 | 0.44 | 0.41 |
| Low Dose | | | | | | | | |

TABLE 13
% Body weight change of each group over study days using
body weight on Day 0 and Day 1 as initial point

| Group | Day −1 | Day 0 | Day 1 | Day 2 | Day 3 | Day 4 | Day 5 | Day 6 |
|---|---|---|---|---|---|---|---|---|
| Group 1 CTP- | 0.68% | 0.00% | −1.15% | −2.30% | −4.60% | 2.30% | 3.45% | 5.75% |
| R8-PLP - High | | | | | | | | |
| Dose | | | | | | | | |
| Group 2 CTP- | −0.78% | 0.00% | 0.78% | 1.48% | 1.69% | 2.99% | 2.64% | 2.39% |
| R8-PLP - | | | | | | | | |
| Medium Dose | | | | | | | | |
| Group 3 CTP- | −1.49% | 0.00% | −0.21% | 0.65% | −2.49% | 0.38% | 1.36% | 0.51% |
| R8-PLP - Low | | | | | | | | |
| Dose | | | | | | | | |
| Group 4 MC3 | −0.30% | 0.00% | −6.09% | −5.90% | −10.19% | −4.11% | −1.35% | 1.48% |
| lipid - High Dose | | | | | | | | |
| Group 5 MC3 | −0.99% | 0.00% | −4.90% | −0.71% | −2.41% | 0.76% | 1.31% | 2.42% |
| lipid - Medium | | | | | | | | |
| Dose | | | | | | | | |
| Group 6 MC3 | 0.61% | 0.00% | 1.61% | 2.84% | 0.65% | 4.29% | 2.36% | 3.26% |
| lipid - Low Dose | | | | | | | | |

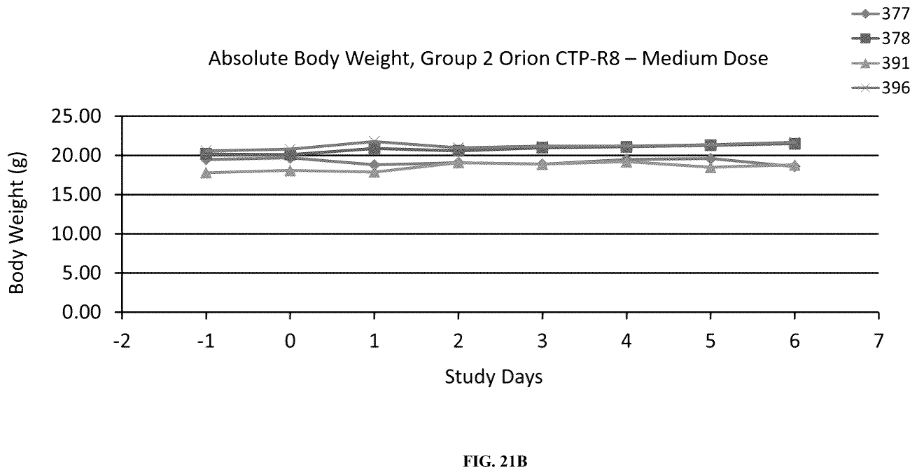

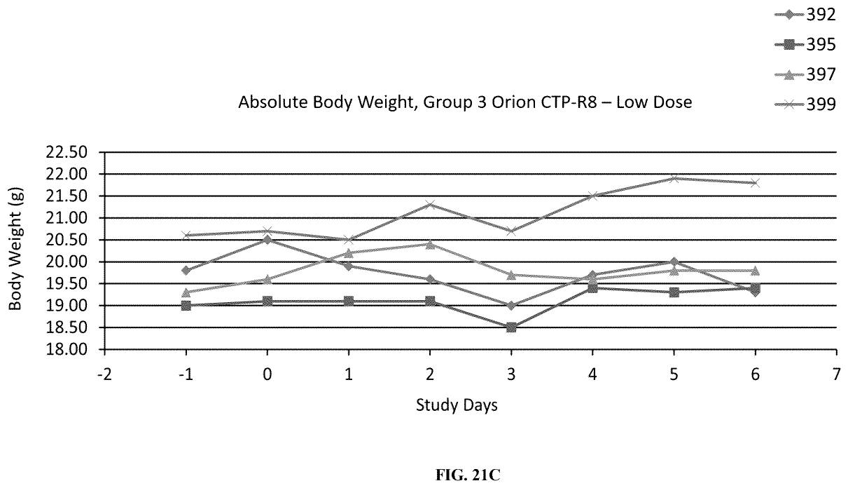

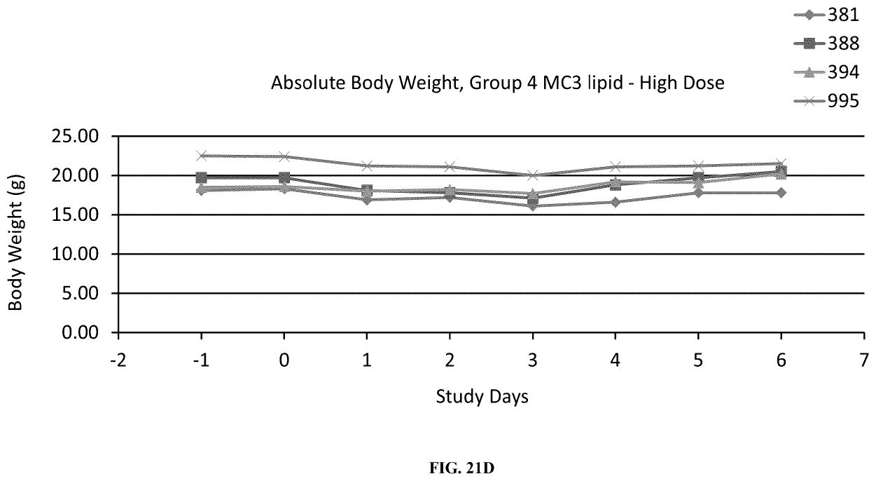

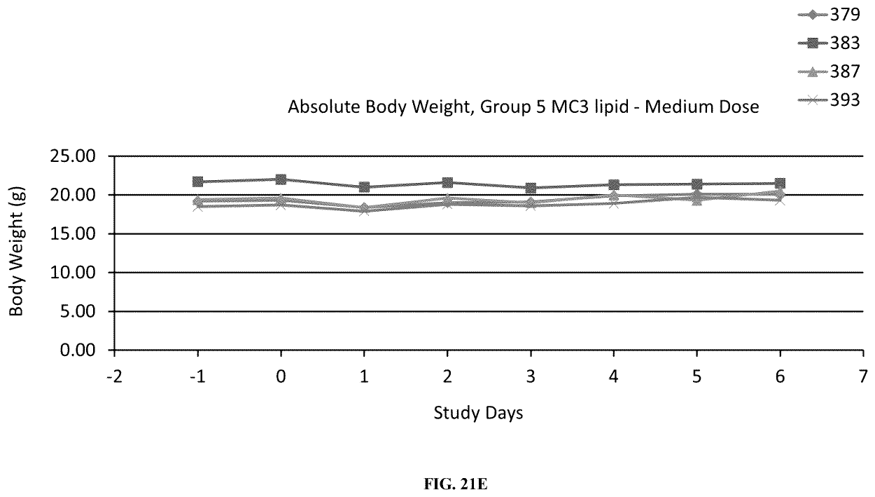

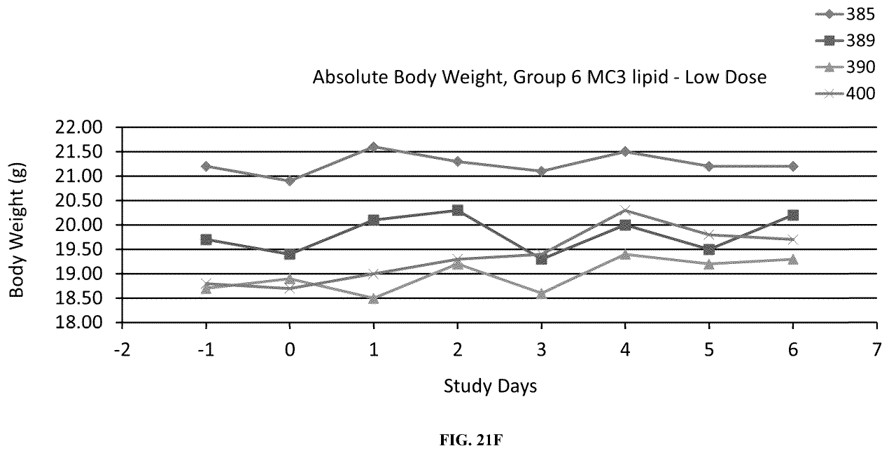

FIGS. 21A-21F depict body weight for each member in the treatment groups 1 to 6, respectively. Table 14 lists body weight of each member in the treatment groups 1-6 per study day. As illustrated in FIGS. 21A and 21B, the treatment groups that received high and medium doses of CTP-R8-PLP (i.e., groups 1 and 2) remained stable body weight. Similarly, FIGS. 21D and 21E illustrate the treatment groups that received high and medium doses of MC3 (i.e., groups 4 and 5) also remained stable body weight. The groups received low doses of CTP-R8-PLP or MC3 showed a larger body weight fluctuation (see FIGS. 21C and 21F).

TABLE 14
Body weight of each member in treatment groups 1-6 per study day

| | Temp | Animal | | Fate | |
|---|---|---|---|---|---|
| Group | ID | ID | Sex | Code | Day |
| Group 1 CTP-R8-PLP-High Dose | 001 | 376 | F | FD | 0 |
| Group 1 CTP-R8-PLP-High Dose | 005 | 380 | F | FD | 0 |
| Group 1 CTP-R8-PLP-High Dose | 007 | 382 | F | FD | 0 |
| Group 1 CTP-R8-PLP-High Dose | 009 | 384 | F | A | |
| Group 2 CTP-R8-PLP-Medium Dose | 002 | 377 | F | A | |
| Group 2 CTP-R8-PLP-Medium Dose | 003 | 378 | F | A | |
| Group 2 CTP-R8-PLP-Medium Dose | 016 | 391 | F | A | |
| Group 2 CTP-R8-PLP-Medium Dose | 021 | 396 | F | A | |
| Group 3 CTP-R8-PLP-Low Dose | 017 | 392 | F | A | |
| Group 3 CTP-R8-PLP-Low Dose | 020 | 395 | F | A | |
| Group 3 CTP-R8-PLP-Low Dose | 022 | 397 | F | A | |
| Group 3 CTP-R8-PLP-Low Dose | 024 | 399 | F | A | |
| Group 4 MC3 lipid-High Dose | 006 | 381 | F | A | |
| Group 4 MC3 lipid-High Dose | 013 | 388 | F | A | |
| Group 4 MC3 lipid-High Dose | 019 | 394 | F | A | |
| Group 4 MC3 lipid-High Dose | 026 | 995 | F | A | |
| Group 5 MC3 lipid-Medium Dose | 004 | 379 | F | A | |
| Group 5 MC3 lipid-Medium Dose | 008 | 383 | F | A | |
| Group 5 MC3 lipid-Medium Dose | 012 | 387 | F | A | |
| Group 5 MC3 lipid-Medium Dose | 018 | 393 | F | A | |
| Group 6 MC3 lipid-Low Dose | 010 | 385 | F | A | |
| Group 6 MC3 lipid-Low Dose | 014 | 389 | F | A | |
| Group 6 MC3 lipid-Low Dose | 015 | 390 | F | A | |
| Group 6 MC3 lipid-Low Dose | 025 | 400 | F | A | |
| FD: Found Dead | | | | | |
| F: Female | | | | | |

| | Study Day | | | | | | | |
|---|---|---|---|---|---|---|---|---|
| Group | −1 | 0 | 1 | 2 | 3 | 4 | 5 | 6 |
| Group 1 CTP-R8-PLP-High Dose | 19.40 | 19.60 | | | | | | |
| Group 1 CTP-R8-PLP-High Dose | 20.00 | 20.20 | | | | | | |
| Group 1 CTP-R8-PLP-High Dose | 20.90 | 20.40 | | | | | | |
| Group 1 CTP-R8-PLP-High Dose | 17.80 | 17.40 | 17.20 | 17.00 | 16.60 | 17.80 | 18.00 | 18.40 |
| Group 2 CTP-R8-PLP-Medium Dose | 19.50 | 19.70 | 18.80 | 19.10 | 18.90 | 19.50 | 19.60 | 18.60 |
| Group 2 CTP-R8-PLP-Medium Dose | 20.20 | 20.10 | 20.90 | 20.60 | 21.00 | 21.10 | 21.30 | 21.50 |
| Group 2 CTP-R8-PLP-Medium Dose | 17.80 | 18.10 | 17.90 | 19.10 | 18.90 | 19.20 | 18.50 | 18.80 |
| Group 2 CTP-R8-PLP-Medium Dose | 20.60 | 20.80 | 21.80 | 21.00 | 21.20 | 21.20 | 21.40 | 21.70 |
| Group 3 CTP-R8-PLP-Low Dose | 19.80 | 20.50 | 19.90 | 19.60 | 19.00 | 19.70 | 20.00 | 19.30 |
| Group 3 CTP-R8-PLP-Low Dose | 19.00 | 19.10 | 19.10 | 19.10 | 18.50 | 19.40 | 19.30 | 19.40 |
| Group 3 CTP-R8-PLP-Low Dose | 19.30 | 19.60 | 20.20 | 20.40 | 19.70 | 19.60 | 19.80 | 19.80 |
| Group 3 CTP-R8-PLP-Low Dose | 20.60 | 20.70 | 20.50 | 21.30 | 20.70 | 21.50 | 21.90 | 21.80 |
| Group 4 MC3 lipid-High Dose | 18.10 | 18.30 | 16.90 | 17.20 | 16.10 | 16.60 | 17.80 | 17.80 |
| Group 4 MC3 lipid-High Dose | 19.70 | 19.70 | 18.10 | 17.80 | 17.10 | 18.80 | 19.70 | 20.50 |
| Group 4 MC3 lipid-High Dose | 18.50 | 18.60 | 18.00 | 18.20 | 17.70 | 19.20 | 19.10 | 20.20 |
| Group 4 MC3 lipid-High Dose | 22.50 | 22.40 | 21.20 | 21.10 | 20.00 | 21.10 | 21.20 | 21.50 |
| Group 5 MC3 lipid-Medium Dose | 19.20 | 19.30 | 18.40 | 19.00 | 19.10 | 19.90 | 20.10 | 20.10 |
| Group 5 MC3 lipid-Medium Dose | 21.70 | 22.00 | 21.00 | 21.60 | 20.90 | 21.30 | 21.40 | 21.50 |
| Group 5 MC3 lipid-Medium Dose | 19.40 | 19.60 | 18.40 | 19.60 | 19.00 | 20.00 | 19.30 | 20.50 |
| Group 5 MC3 lipid-Medium Dose | 18.50 | 18.70 | 17.90 | 18.80 | 18.60 | 18.90 | 19.70 | 19.30 |
| Group 6 MC3 lipid-Low Dose | 21.20 | 20.90 | 21.60 | 21.30 | 21.10 | 21.50 | 21.20 | 21.20 |
| Group 6 MC3 lipid-Low Dose | 19.70 | 19.40 | 20.10 | 20.30 | 19.30 | 20.00 | 19.50 | 20.20 |
| Group 6 MC3 lipid-Low Dose | 18.70 | 18.90 | 18.50 | 19.20 | 18.60 | 19.40 | 19.20 | 19.30 |
| Group 6 MC3 lipid-Low Dose | 18.80 | 18.70 | 19.00 | 19.30 | 19.40 | 20.30 | 19.80 | 19.70 |

### Clinical Chemistry:

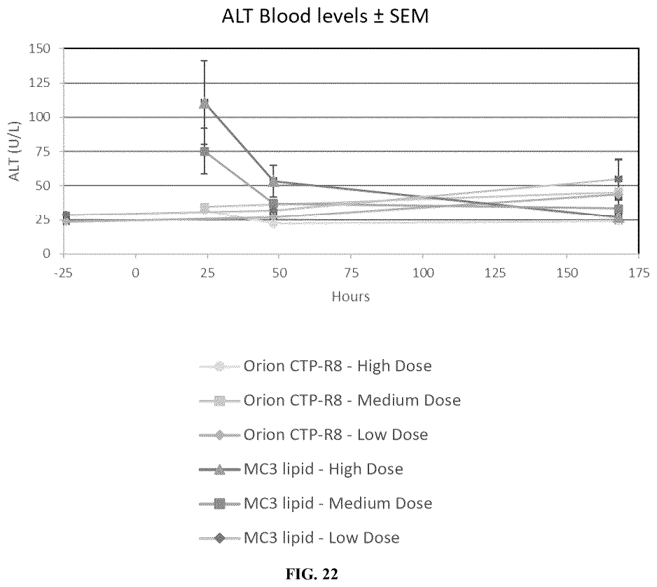

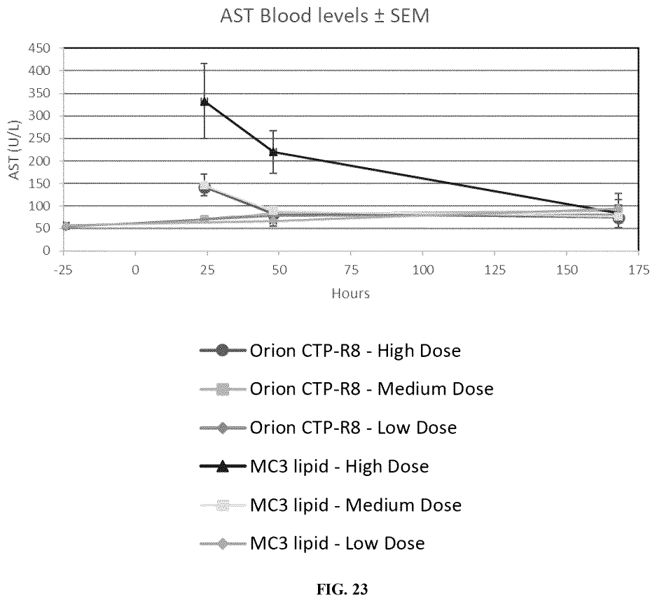

FIG. 22 illustrates ALT measurement of the treatment groups. The treatment groups that received high and medium doses of MC3 had a substantial increase in the ALT concentration approximately 25 hours after receiving the control articles. All the treatment groups remained a relatively stable ALT concentration after approximately 170 hours. FIG. 23 illustrates AST measurement of the treatment groups. The treatment groups that received high and medium doses of MC3 as well the group that received low dose of CTP-R8-PLP had a substantial increase in the AST concentration approximately 25 hours after receiving the control articles. All the treatment groups remained a relatively stable AST concentration after approximately 170 hours.

All data in the above Examples were reported as mean±SEM. Statistical analyses were performed using Student's t-test or one-way ANOVA and a post-hoc Student-Newman-Keuls test using SigmaStat 3.5 software. Probability (P) values <0.05 are considered to be significant.

What is claimed is:

1. A lipid nanoparticle, comprising:
a lipid layer, wherein the lipid layer comprises a helper lipid, a PEG lipid, a structural lipid, or a combination thereof;
a cell-penetrating peptide conjugated to the lipid layer;
a collagen-targeting peptide conjugated to the lipid layer; and
a nucleic acid associated with a vascular disease or condition.

2. The lipid nanoparticle of claim 1, wherein the nucleic acid is encapsulated in the lipid nanoparticle.

3. The lipid nanoparticle of claim 1 or 2, wherein the nucleic acid comprises a DNA.

4. The lipid nanoparticle of claim 1 or 2, wherein the nucleic acid comprises an RNA, optionally an siRNA.

5. The lipid nanoparticle of claim 4, wherein a wt/wt ratio of total lipids to the nucleic acid in the nanoparticle is about 10:1 to about 150:1.

6. The lipid nanoparticle of any one of claims 1 to 5, wherein the nucleic acid is associated with matrix metalloproteinase or other vascular remodeling pathways.

7. A lipid nanoparticle, comprising:
a lipid layer, wherein the lipid layer comprises a helper lipid, a PEG lipid, a structural lipid, or a combination thereof;
a cell-penetrating peptide conjugated to the lipid layer;
a collagen-targeting peptide conjugated to the lipid layer; and
a biologically active ingredient that comprises one or more of antibiotics, stimulants, statins, b-receptor blockers, anti-hypertensives, anticoagulants, bronchodilators, corticosteroids, insulin, vaccines, immunosuppressants, interferons, antibodies, proteins, and peptides.

8. The lipid nanoparticle of any one of claims 1 to 7, wherein the collagen-targeting peptide is conjugated with a lipid to form a collagen-targeting lipid.

9. The lipid nanoparticle of claim 8, wherein the collagen-targeting lipid comprises about 0.5 mol % to about 15 mol % of the total lipid in the nanoparticle.

10. The lipid nanoparticle of claim 8, wherein the collagen-targeting lipid comprises about 5 mol % to about 10 mol % of the total lipid in the nanoparticle.

11. The lipid nanoparticle of any one of claims 8 to 10, wherein the collagen-targeting lipid comprises a PEG lipid.

12. The lipid nanoparticle of any one of claims 8 to 11, wherein the collagen-targeting lipid comprises pegylated 1,2-distearoyl-sn-glycero-3-phosphoethanolamine (DSPE).

13. The lipid nanoparticle of any one of claims 1 to 12, wherein the collagen-targeting peptide comprises a sequence of KLWVLPK, KIWKLPQ, KIFVWPY, KVWSLPQ, RRANAALKAGELYKCILY, RRANAALKAGELYKSILYGC, TKKTLRT, or WREPSFMALS.

14. The lipid nanoparticle of any one of claims 1 to 12, wherein the collagen-targeting peptide comprises a sequence of KLWVLPK.

15. The lipid nanoparticle of any one of claims 1 to 14, wherein the cell-penetrating peptide is conjugated with a lipid or a hydrophobic moiety to form a cell-penetrating lipid.

16. The lipid nanoparticle of claim 15, wherein the cell-penetrating lipid comprises about 0.5 mol % to about 15 mol % of the total lipid in the nanoparticle.

17. The lipid nanoparticle of claim 15, wherein the cell-penetrating lipid comprises about 5 mol % to about 10 mol % of the total lipid in the nanoparticle.

18. The lipid nanoparticle of any one of claims 15 to 17, wherein the cell-penetrating peptide is conjugated with a lipid to form the cell-penetrating lipid.

19. The lipid nanoparticle of any one of claims 15 to 17, wherein the cell-penetrating peptide is conjugated with a PEG lipid (e.g., pegylated DSPE) to form the cell-penetrating lipid.

20. The lipid nanoparticle of any one of claims 15 to 17, wherein the cell-penetrating peptide is conjugated with a hydrophobic moiety to form the cell-penetrating lipid.

21. The lipid nanoparticle of claim 20, wherein the hydrophobic moiety is a fatty acid, fatty alcohol, or fatty ester.

22. The lipid nanoparticle of claim 20, wherein the cell-penetrating peptide is covalently attached to a stearic acid to form the cell-penetrating lipid.

23. The lipid nanoparticle of any one of claims 1 to 22, wherein the cell-penetrating peptide comprises polyarginine, poly-L-lysine, poly-L-histidine, penetratin and derivatives thereof, MPG peptide, Pep-1 peptide, CADY peptide, KALA peptide, HA2 peptide, histones, poly-β-amino acid esters, or combinations thereof.

24. The lipid nanoparticle of claim 23, wherein the cell-penetrating peptide comprises polyarginine, polylysine, or polyhistidine.

25. The lipid nanoparticle of claim 24, wherein the cell-penetrating peptide comprises polyarginine.

26. The lipid nanoparticle of claim 25, wherein the polyarginine is octaarginine (R8).

27. The lipid nanoparticle of claim 26, wherein the cell-penetrating peptide is conjugated with stearic acid to form a cell-penetrating lipid, and wherein the cell-penetrating lipid is STR-R8.

28. The lipid nanoparticle of any one of claims 1 to 27, further comprising at least one of a monovalent cation or a multivalent cation.

29. The lipid nanoparticle of claim 28, wherein the monovalent cation or multivalent cation is a divalent cation.

30. The lipid nanoparticle of claim 29, wherein the divalent cation is selected from the group consisting of calcium (Ca2+), magnesium (Mg2+), ferrous (Fe2+), and combinations thereof.

31. The lipid nanoparticle of claim 30, wherein the divalent cation is calcium (Ca2+).

32. The lipid nanoparticle of any one of claims 28 to 31, wherein the monovalent cation or the multivalent cation is present at a concentration of about 5 mM to about 50 mM.

33. The lipid nanoparticle of any one of claims 28 to 31, wherein the monovalent cation or the multivalent cation is present at a concentration of about 10 mM to about 40 mM.

34. The lipid nanoparticle of any one of claims 28 to 31, wherein the monovalent cation or the multivalent cation is present at a concentration of about 10 mM to about 15 mM.

35. The lipid nanoparticle of any one of claims 1 to 34, wherein the lipid nanoparticle comprises a helper lipid.

36. The lipid nanoparticle of claim 35, wherein the helper lipid is a phospholipid.

37. The lipid nanoparticle of claim 35 or 36, wherein the helper lipid is 1,2-dioleoyl-sn-glycero-3-phosphocholine (DOPC), 1,2-distearoyl-sn-glycero-3-phosphoethanolamine (DSPE), 1,2-distearoyl-sn-glycero-3-phosphocholine (DSPC), 1,2-dipalmitoyl-sn-glycero-3-phosphocholine (DPPC), 1,2-dimyristoyl-sn-glycero-phosphocholine (DMPC), 2-oleoyl-1-palmitoyl-sn-glycero-3-phosphocholine (POPC), 1,2-dioleoyl-sn-glycero-3-phosphoethanolamine (DOPE), sphingomyelin, phosphatidyl inositol (PI), or phosphatidic acid (PA).

38. The lipid nanoparticle of claim 35, wherein the helper lipid is DOPC.

39. The lipid nanoparticle of any one of claims 1 to 38, wherein the helper lipid comprises about 10 mol % to about 80 mol % of the total lipid in the nanoparticle.

40. The lipid nanoparticle of any one of claims 1 to 39, wherein the lipid nanoparticle comprises a PEG lipid.

41. The lipid nanoparticle of claim 40, wherein the PEG lipid comprises PEG-modified phosphatidylethanolamines, PEG-modified phosphatidic acids, PEG-modified ceramides, PEG-modified dialkylamines, PEG-modified diacylglycerols, PEG-modified dialkylglycerols, and a mixture thereof.

42. The lipid nanoparticle of claim 40, wherein the PEG lipid comprises PEG-dilauroylglycerol, PEG-dimyristoylglycerol (PEG-DMG), PEG-dipalmitoylglycerol, PEG-distearoylglycero-phosphoethanolamine (PEG-DSPE), PEG-dipalmitoylglycerol, PEG-disterylglycerol, PEG-dilaurylglycamide, PEG-dimyristylglycamide, PEG-dipalmitoylglycamide, PEG-disterylglycamide, PEG-cholesterol, and PEG-DMB (3,4-Ditetradecoxylbenzyl-[omega]-methyl-poly(ethylene glycol) ether), 1,2-dimyristoyl-sn-glycero-3-phosphoethanolamine-N-[methoxy(polyethylene glycol)-2000]), PEG-DOPC, or a mixture thereof.

43. The lipid nanoparticle of claim 40, wherein the PEG lipid is 1,2-distearoyl-sn-glycero-3-phosphoethanolamine-N-[methoxy(polyethyleneglycol)-2000](DSPE-PEG(2000)).

44. The lipid nanoparticle of any one of claims 1 to 43, wherein the PEG lipid comprises about 1 mol % to about 20 mol % of the total lipid in the nanoparticle.

45. The lipid nanoparticle of any one of claims 1 to 44, wherein the lipid nanoparticle comprises a structural lipid.

46. The lipid nanoparticle of claim 45, wherein the structural lipid comprises steroid, sterol, alkyl resoreinol, cholesterol or derivative thereof, fecosterol, sitosterol, ergosterol, campesterol, stigmasterol, brassicasterol, tomatidine, tomatine, ursolic acid, alpha-tocopherol, or a combination thereof.

47. The lipid nanoparticle of claim 45, wherein the structural lipid is cholesterol.

48. The lipid nanoparticle of any one of claims 1 to 47, wherein the structural lipid comprises about 5 mol % to about 30 mol % of the total lipid in the nanoparticle.

49. A method of treating a vascular disease in a subject in need thereof, comprising administering to the subject a lipid nanoparticle of any one of claims 1 to 48.

50. A method of treating vascular disease in a subject in need thereof, comprising:
administering to the subject a lipid nanoparticle comprising:
a lipid layer;
a cell-penetrating peptide conjugated to the lipid layer,
a collagen-targeting peptide conjugated to the lipid layer, and
a therapeutically effective ingredient encapsulated in the lipid nanoparticle.

51. The method of claim 50, wherein the therapeutically effective ingredient comprises RNA or DNA.

52. The method of claim 50 or 51, wherein a wt/wt ratio of total lipids to the therapeutically effective ingredient in the nanoparticle is about 10:1 to about 150:1.

53. The method of any one of claims 49-52, wherein the subject's blood alanine aminotransferase (ALT) level does not fluctuate more than 50% at 25 hours after the administering.

54. The method of any one of claims 49-53, wherein the subject's blood aspartate aminotransferase (AST) level does not fluctuate more than 50% at 25 hours after the administering.

55. A method of preparing lipid nanoparticles, comprising:
(a) combining a hydrophobic mixture with an aqueous solution, wherein the hydrophobic mixture comprises:
one or more lipids, wherein the one or more lipids comprise a helper lipid, a PEG lipid, a structural lipid, or a combination thereof,
a cell-penetrating peptide, and
a collagen-targeting peptide, and
wherein the aqueous solution comprises a cation and a biological active ingredient, wherein the biological active ingredient is associated with a vascular disease or condition
thereby forming the lipid nanoparticle; and
(b) isolating the lipid nanoparticles.

56. The method of claim 55, wherein the biological active ingredient comprises nucleic acid.

57. The method of claim 55, wherein the nucleic acid comprises an siRNA.

58. The method of any one of claims 55 to 57, wherein the cation comprises at least one of a monovalent cation or a multivalent cation.
